(19) United States
(12) Patent Application Publication
(10) Pub. No.: US 2023/0048936 A1
(43) Pub. Date: Feb. 16, 2023

(54) SYSTEM AND METHOD FOR DETECTING PRESENCE OF A TARGET BIOPARTICLE IN A SAMPLE VIA A VERTICAL FLOW ASSAY

(71) Applicant: KAYA17 INC., Pleasanton, CA (US)

(72) Inventors: Srinagesh Satyanarayana, Pleasanton, CA (US); Sulatha Dwarakanath, Pleasanton, CA (US); Gokul Kowligi, Pleasanton, CA (US); Anup Madan, Pleasanton, CA (US)

(21) Appl. No.: 17/894,970

(22) Filed: Aug. 24, 2022

Related U.S. Application Data
(63) Continuation-in-part of application No. 16/825,801, filed on Mar. 20, 2020.
Application No. 16/825,801 is a continuation-in-part of application No. 16/690,589, filed on Nov. 21, 2019.
(60) Provisional application No. 63/348,270, filed on Jun. 2, 2022.
(60) Provisional application No. 63/238,970, filed on Aug. 31, 2021.
(60) Provisional application No. 63/236,301, filed on Aug. 24, 2021.

(30) Foreign Application Priority Data
Nov. 22, 2018 (IN) 201841044093
Mar. 22, 2019 (IN) 201941011177

(51) Int. Cl. B01D 61/14; G01N 21/64; G01N 33/543; B01L 3/00

(52) U.S. Cl. CPC B01D 61/147; G01N 21/6428; G01N 33/54313; B01L 3/502738; B01L 3/502715; G01N 2021/6439; B01L 2200/16

(57) ABSTRACT

One variation of a system includes a cartridge comprising: a substrate; a sample well integrated into the substrate, defining an upper opening and a lower opening, and configured to receive a test solution comprising a user sample and an amount of a fluorescent probe configured to bind with a target bioparticle to form a target complex; a filter membrane extending across the lower opening and defining a network of pores configured to convey fluid from the sample well and prevent passage of the target complex through the filter membrane. The system further includes a reader comprising: a housing; a cartridge receptacle configured to receive the cartridge; an excitation source configured to illuminate a detection region within the housing; and a detector defining a field of view intersecting the detection region and configured to detect a signal generated by fluid in the sample well and representing presence of the target bioparticle.

### CROSS-REFERENCE TO RELATED APPLICATIONS

This application claims the benefit of U.S. Provisional Application No. 63/348,270, filed on 2 Jun. 2022, U.S. Provisional Application No. 63/238,970, filed on 31 Aug. 2021, and U.S. Provisional Application No. 63/236,301, filed on 24 Aug. 2021, each of which is incorporated in its entirety by this reference.

This application is a continuation-in-part application of U.S. patent application Ser. No. 16/825,801, filed on 20 Mar. 2020, which claims the benefit of Indian Patent Application No. 201941011177, filed on 22 Mar. 2019, each of which are incorporated in their entireties by this reference. This application is also a continuation-in-part application of U.S. patent application Ser. No. 16/690,589, filed on 21 Nov. 2019, which claims the benefit of Indian Patent Application No. 201841044093, filed on 22 Nov. 2018, each of which are incorporated in their entireties by this reference.

### TECHNICAL FIELD

This invention relates generally to the field of diagnostic detection and more specifically to a new and useful system and method for a vertical flow assay in the field of diagnostic detection.

### BRIEF DESCRIPTION OF THE FIGURES

FIG. 1 is a schematic representation of a system;

FIGS. 2A and 2B are schematic representations of the system;

FIGS. 3A and 3B are schematic representations of the system;

FIGS. 4A, 4B, and 4C are schematic representations of the system;

FIGS. 5A, 5B, and 5C are schematic representations of the system;

FIG. 6 is a schematic representation of a system;

FIGS. 7A and 7B are schematic representations of the system;

FIG. 8 is a schematic representation of the system;

FIGS. 9A and 9B are schematic representations of the system; and

FIGS. 10A and 10B are schematic representations of the system.

### DESCRIPTION OF THE EMBODIMENTS

The following description of embodiments of the invention is not intended to limit the invention to these embodiments but rather to enable a person skilled in the art to make and use this invention. Variations, configurations, implementations, example implementations, and examples described herein are optional and are not exclusive to the variations, configurations, implementations, example implementations, and examples they describe. The invention described herein can include any and all permutations of these variations, configurations, implementations, example implementations, and examples.

### 1. System: Cartridge+Reader

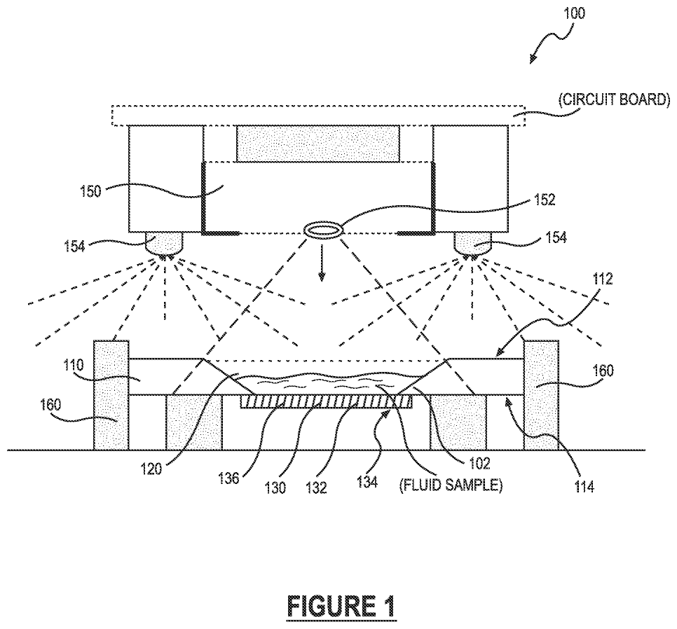

As shown in FIGS. 1-10B, a system 100 includes a cartridge 102 and a reader 150.

The cartridge 102 includes a substrate 110 and a filter membrane 130.

The substrate 110 defines: an upper surface 112 defining an upper opening 122; a lower surface 114, opposite the upper surface 112, defining a lower opening 124; and a sample well 120—extending between the upper opening 122 and the lower opening 124—configured to receive a test solution including a user sample mixed with a probe solution including an amount of a fluorescent probe configured to bind with a target bioparticle to form a target complex.

The filter membrane 130 is coupled to the lower surface 114 of the substrate 110 and extends across the lower opening 124. The filter membrane 130 defines: an inner surface 132 facing the sample well 120 and configured to receive fluid and particulate from the sample well 120; an outer surface 134; and a network of pores 136—extending between the inner surface 132 and the outer surface 134—configured to convey fluid and biological particulate from the sample well 120 through the filter membrane 130 and prevent passage of the target complex through the filter membrane 130.

The reader 150 includes: a housing 192; a cartridge receptacle 160 configured to transiently receive the cartridge 102 to locate the sample well 120 within a detection region within the housing 192; an excitation source 154 arranged proximal the cartridge receptacle 160 within the housing 192 and configured to illuminate the detection region according to a target excitation intensity; and a detector 152 arranged within the housing 192, defining a field of view intersecting the detection region, and configured to detect an optical signal—generated by fluid in the sample well 120—representing presence of the target bioparticle in fluid in the sample well 120.

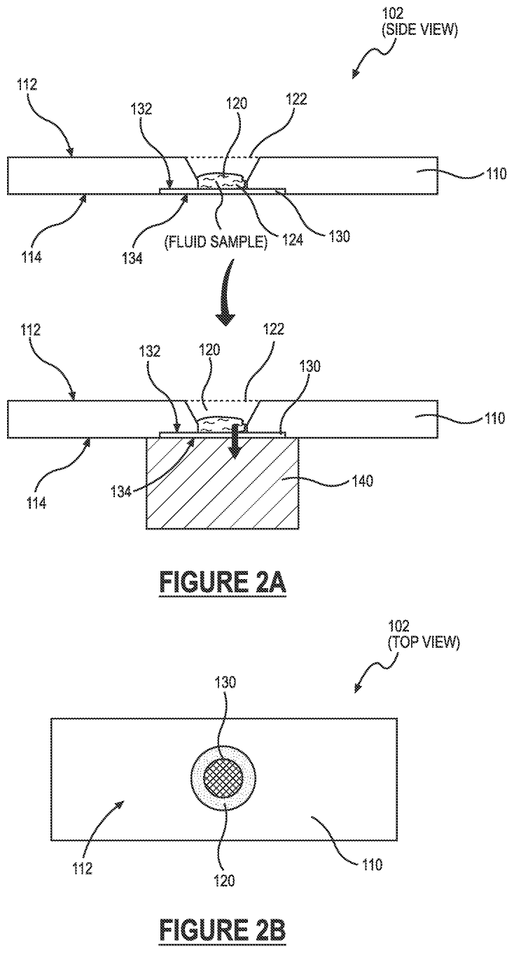

In one variation, as shown in FIG. 2A, the system 100 further includes an absorbent pad stack 140: removably coupled to the outer surface 134 of the filter membrane 130; and configured to cooperate with the filter membrane 130 to draw fluid and biological particulate (e.g., unbound fluorescent probe) from the inner surface 132 of the filter membrane 130, through the network of pores 136, and onto the absorbent pad stack 140.

One variation of the system 100 includes the cartridge 102 including the substrate 110 defining: an upper surface 112 defining an upper opening 122; a lower surface 114 defining a lower opening 124; and a sample well 120 extending between the upper opening 122 and the lower opening 124 and configured to receive a user sample and a probe solution including an amount of a fluorescent probe of a first size and configured to bind with a target bioparticle to form a target complex of a second size. In this variation, the cartridge 102 includes a filter membrane 130: extending across the lower opening 124 of the sample well 120; and defining a network of pores 136, within a target pore size range, configured to convey fluid and biological particulate from the sample well 120 through the filter membrane 130 and prevent passage of the target complex through the filter membrane 130, pore sizes in the target pore size range exceeding the first size and less than the second size. In this variation, the reader 150 includes: a housing 192; a cartridge receptacle 160 configured to receive the cartridge 102 and locate the sample well 120 within a detection region within the housing 192; an excitation source 154 arranged proximal the cartridge receptacle 160 within the housing 192 and configured to illuminate the detection region; a detector 152 arranged within the housing 192; defining a field of view intersecting the detection region, and configured to detect an optical signal generated by contents of the sample well 120 responsive to illumination of the sample well 120 via the excitation source 154. In one variation, the system 100 can further include a controller 190—coupled to the reader 150—configured to interpret presence of the target bioparticle in the sample well 120 based on the optical signal.

### 1.1 System: Cartridge+Detection Module

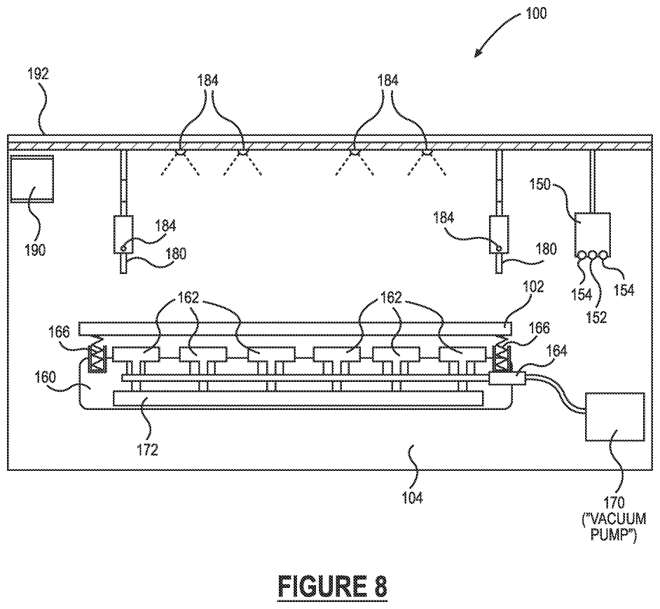

As shown in FIGS. 8, 9A, 9B, 10A, and 10B, one variation of the system 100 includes a cartridge 102 and a detection module 104.

In this variation, the cartridge 102 includes: a substrate 110 defining an upper surface 112 and a lower surface 114; a set of sample wells 120 integrated into the substrate 110, each sample well 120 in the set of sample wells 120 defining an upper opening 122, in a set of upper openings 122, arranged on the upper surface 112, defining a lower opening 124, in a set of lower openings 124, arranged on the lower surface 114; and including a filter membrane 130, in a set of filter membranes 130. In this variation, each filter membrane 130 in the set of filter membranes 130: is coupled to the lower surface 114, extending across the lower opening 124, and defines: an inner surface 132 facing the sample well 120; an outer surface 134; and a network of pores 136 extending between the inner surface 132 and the outer surface 134 and configured to promote transfer of fluid and biological particulate from the inner 132 surface to the outer surface 134 and inhibit passage of a target complex through the filter membrane 130, the target complex including a fluorescent probe, in a set of fluorescent probes, bound to a target bioparticle, in a set of target bioparticles.

In this variation, the detection module 104 includes: a cartridge receptacle 160 configured to receive the cartridge 102 and including a platform 162 defining a set of apertures 163 and configured to support the cartridge 102 in a drain position and a set of pump inlets 164 fluidly coupled to the set of apertures 163; a set of fluid dispensers 180 arranged above the cartridge receptacle 160 and configured to dispense metered volumes of fluid into the set of sample wells 120; a set of pumps 170 (e.g., a vacuum pump and/or peristaltic pump) coupled to the set of pump inlets 164 and configured to draw fluid and biological particulate from the inner surface 132 of the filter membrane 130 through the network of pores 136, and away from the outer surface 134 of the filter membrane 130; and a reader 150 (e.g., including a detector 152 and an excitation source 154) arranged above the cartridge receptacle 160 and configured to detect a set of optical signals generated by fluid in the set of sample wells 120 responsive to excitation of the set of fluorescent probes, the set of optical signals representing presence of the set of target bioparticles in the set of sample wells 120; and a controller 190 configured to selectively trigger actuation of the set of fluid dispensers 180, the set of pumps 170, and the reader 150.

In one variation, the system 100 further includes a waste reservoir 172 arranged below the platform 162 (e.g., below a base surface of the platform 162) and configured to collect fluid and biological particulate released from the outer surface 134 of the filter membrane 130.

One variation of the system 100 includes the cartridge 102 and the detection module 104. In this variation, the cartridge 102 includes: a substrate 110 defining an upper surface 112 and a lower surface 114; a first sample well 120 defining an upper opening 122 arranged on the upper surface 112, defining a lower opening 124 arranged on the lower surface 114 and including a filter membrane 130. The filter membrane 130: is coupled to the lower surface 114; extends across the lower opening 124; and defines: an inner surface 132 facing the first sample well 120, an outer surface 134, and a network of pores 136 extending between the inner surface 132 and the outer surface 134 and configured to promote transfer of fluid and biological particulate from the inner surface 132 to the outer surface 134 and inhibit passage of a target complex through the filter membrane 130, the target complex including a fluorescent probe bound to a target bioparticle. In this variation, the detection module 104 includes: a cartridge receptacle 160 configured to receive the cartridge 102 and including: a platform 162 defining a set of apertures 163 and configured to contact the lower surface 114 to support the cartridge 102 in a drain position; a set of pump inlets 164 fluidly coupled to the array of apertures 163; a set of fluid dispensers arranged above the cartridge receptacle and configured to dispense metered volumes of fluid into the first sample well 120; a set of pumps 170 coupled to the set of pump inlets 164 and configured to draw fluid and biological particulate from the inner surface 132 of the filter membrane 130 through the network of pores 136, and away from the outer surface 134 of the filter membrane 130; a reader 150 (e.g., including a detector 152 and an excitation source 154) arranged above the cartridge receptacle 160 and configured to detect an optical signal generated by fluid in the first sample well 120 responsive to excitation of the fluorescent probe; and a controller 190 configured to selectively trigger actuation of the set of fluid dispensers, the set of pumps 170, and the reader according to a predefined assay and interpret presence of the target bioparticle in the first sample well 120 based on the optical signal.

One variation of the cartridge includes: a substrate 110 defining an upper surface 112 and a lower surface 114; and a set of sample wells 120, each sample well 120, in the set of sample wells 120 defining an upper opening 122 arranged on the upper surface 112, defining a lower opening 124 arranged on the lower surface 114, and including a filter membrane 130 coupled to the lower surface 114 and extending across the lower opening 124. The filter membrane 130 defines: an inner surface 132 facing the sample well 120; an outer surface 134; and a network of pores 136 extending between the inner surface 132 and the outer surface 134 and configured to promote transfer of fluid and biological particulate from the inner surface 132 to the outer surface 134 and inhibit passage of a target complex through the filter membrane 130, the target complex including a fluorescent probe bound to a target bioparticle. In this variation, the detection module 104 includes: a cartridge receptacle 160 configured to receive the cartridge 102 and defining a set of apertures 163; a set of fluid dispensers 180 arranged above the cartridge receptacle 160 and configured to dispense metered volumes of fluid into the first sample well 120; a set of pumps 170 fluidly coupled to the set of apertures 163 and configured to draw fluid and biological particulate from the inner surface 132 of the filter membrane 130 through the network of pores 136, and away from the outer surface 134 of the filter membrane 130 of each sample well 120 in the set of sample wells 120; and a reader 150 arranged above the cartridge receptacle 160. In this variation, the reader 150 includes: an excitation source 152 configured to illuminate a detection region according to a target excitation wavelength; and a detector 154 (e.g., a photodetector) defining a field of view intersecting the detection region and configured to detect an optical signal generated by fluid in a first sample well 120, in the set of sample wells 120, located within the detection region, responsive to activation of the excitation source 154, the optical signal representing presence of the target bioparticle in the first sample well 120.

In one variation, the cartridge 102 includes: a substrate 110 defining an upper surface 112 and a lower surface 114; a sample well 120, in a set of sample wells 120, integrated into the substrate 110 and extending between an upper opening 122 arranged on the upper surface 112 and a lower opening 124 arranged on the lower surface 114; and a filter membrane 130, in a set of filter membrane 130s, coupled to the lower surface 114 and extending across the lower opening 124. The filter membrane 130 defines: an inner surface 132 facing the sample well 120; an outer surface 134; and a network of pores 136 extending between the inner surface 132 and the outer surface 134 and configured to promote transfer of fluid and biological particulate from the inner surface 132 to the outer surface 134 and inhibit passage of a target complex through the filter membrane 130, the target complex including a fluorescent probe bound to a target bioparticle.

In one variation, the detection module 104 includes: a cartridge receptacle 160; a set of fluid dispensers 180; a first pump 170 (e.g., a vacuum pump); a reader 150; and a controller 190. The cartridge receptacle 160: is configured to receive the cartridge 102; includes a platform 162 defining an array of apertures and configured to contact the lower surface 114 to support the cartridge 102 in a drain position; and includes a vacuum inlet fluidly coupled to the array of apertures. The set of fluid dispensers 180 is arranged above the cartridge receptacle 160 and configured to dispense metered volumes of fluid into the set of sample wells 120. The first pump 170 is coupled to the vacuum inlet and configured to apply a vacuum between the cartridge receptacle 160 and the cartridge 102 to draw fluid through the set of filter membranes 130. The reader 150 is arranged above the cartridge receptacle 160 and configured to detect an optical signal generated by fluid in the sample well 120 responsive to excitation of the fluorescent probe. The controller 190 is configured to: coordinate motion of the set of fluid dispensers 180 and the reader 150 according to a position of the sample well 120 on the substrate 110; selectively trigger actuation of the set of fluid dispensers 180, the first pump 170, and the reader 150; and interpret presence of the target bioparticle in the sample well 120 based on the optical signal.

### 2. Method

A method S100 includes, during an incubation period, mixing a first volume of a user sample with a second volume of a probe solution to generate a test solution, the probe solution including an amount of a fluorescent probe configured to bind to a target bioparticle to form a target complex of a target size, and, during a loading period succeeding the incubation period, transferring the test solution into a first sample well 120 arranged on a cartridge 102 including: a substrate 110 defining an upper surface 112 and a lower surface 114; the sample well 120 extending between an upper opening 122 arranged on the upper surface 112 and a lower opening 124 arranged on the lower surface 114; a filter membrane 130 coupled to the lower surface 114, extending across the lower opening 124 and defining an inner surface 132 facing the sample well 120, an outer surface 134, and a network of pores 136 extending between the inner surface 132 and the outer surface 134 and configured to prevent flow of the target complex through the filter membrane 130; and an absorbent pad stack 140 removably coupled to the lower surface 114 of the filter membrane 130 and configured to cooperate with the filter membrane 130 to draw fluid and biological particulate from the inner surface 132, through the network of pores 136, and into the absorbent pad stack 140. The method S100 further includes, during a detection period succeeding the incubation period: at a first time, decoupling the absorbent pad stack 140 from the lower surface 114; at a second time succeeding the first time, loading a volume of a read buffer into the sample well 120 to generate a fluid suspension of particles present on the filter membrane 130 within the sample well 120; detecting a fluorescence signal emitted by the fluid suspension via spectroscopic detection; and characterizing presence of the target bioparticle in the user sample based on the fluorescence signal.

One variation of the method S100 includes: generating a probe solution including an amount of a fluorescent probe in solution, the fluorescent probe including an antibody coupled to a fluorescent tag and configured to bind to the target bioparticle to form a target complex of a target size and generate a fluorescence signal representing presence of the target bioparticle; during an incubation period of a target duration, mixing a first volume of a test solution with a second volume of the fluorescent probe solution in an incubation vessel to generate a conjugated sample; and, during a loading period succeeding the incubation period, transferring the mixture into a sample well 120 configured to receive the mixture at an upper opening 122 of the sample well 120 and directing the conjugated sample vertically downward toward a filter membrane 130 arranged beneath a lower opening 124 of the sample well 120, including pores 136 of sizes within a pore size range, and configured to prevent flow of the target complex through the filter membrane 130 and direct fluid and unbound instances of the fluorescent probe through the filter membrane 130 for collection within an absorbent pad stack 140 removably coupled to the filter membrane 130 and configured to contact the filter membrane 130 opposite the sample well 120 to draw fluid through the filter membrane 130. In this variation, the method S100 further includes, during a detection period succeeding the loading period, decoupling the absorbent pad stack 140 from the cartridge 102, and, in response to decoupling the absorbent pad stack 140 from the cartridge 102, loading a volume of a read buffer to the sample well 120 to generate a suspension of particles present on the filter; detecting a fluorescence signal emitted by the suspension of particles present on the filter membrane 130 via spectroscopic detection; and characterizing presence of the target bioparticle on the filter membrane 130 based on the fluorescence signal.

### 3. Applications

Generally, a system 100 includes a cartridge 102 configured to: receive a user sample (e.g., saliva, blood, serum, urine) mixed with fluorescent probe—configured to selectively bind to a target bioparticle (e.g., a target antigen) to form a target complex (e.g., an antigen-probe complex)—within a sample well arranged on a substrate 110; retain the target complex on a filter membrane 130 arranged across a bottom of the sample well 120; and drain fluid and other biological particulate from the sample well 120, through the filter membrane 130, and onto an absorbent pad stack 140 (e.g., a set of absorbent pads) removably coupled to the filter membrane 130 opposite the sample well. The system 100 further includes a reader 150 configured to: receive the cartridge 102—containing an amount of the target complex suspended in a read buffer added to the sample well—for detection of the target bioparticle in the sample well 120—and therefore in the original user sample—based on detection of an optical signal generated by the fluorescent probe bound to the target bioparticle.

For example, the system 100 can be configured to: process the user sample on the cartridge 102 to isolate the target complex including the target bioparticle; detect an optical signal representing presence of the target bioparticle in the sample well 120 at the reader 150; output a quantitative measure of the target bioparticle load; and interpret presence of the target bioparticle in the user sample—such as a positive result indicating presence above a threshold, a negative result indicating presence below the threshold, and/or a particular concentration of the target bioparticle—for reporting to the user within 15 minutes of receiving the user sample from the user.

In particular, the system 100 can be configured to detect presence of a target bioparticle—such as a specific antigen, a bacteria, a virus, an IGE corresponding to a particular allergen, a protein biomarker (e.g., an ovarian cancer biomarker), a segment of DNA and/or RNA—in a user sample loaded in the sample well 120. More specifically, the reader 150—including an excitation source 154 (e.g., an array of UV LEDs) and a detector 152 (e.g., photodetector), such as including an optical sensor—can be configured to: locate the sample well 120 within a detection region intersecting a field of view defined by the detector 152; uniformly illuminate the sample well 120—according to a target excitation intensity corresponding to the fluorescent probe—via activation of the excitation source 154; and detect an optical signal—filtered according to a target wavelength emission matched to the fluorescent probe bound to the target bioparticle—corresponding to total fluorescence generated within the detection region encompassing the sample well 120 via the detector 152 (e.g., a photodetector). A remote computer system 100 and/or a controller 190 coupled to the reader 150 can then interpret presence of the target bioparticle in the sample well 120 based on the optical signal, such as based on a magnitude (e.g., intensity) of the optical signal.

In one implementation, the cartridge 102 can be configured to include multiple wells arranged across a substrate 110. For example, the cartridge 102 can include: a first sample well 120 arranged in a first position on the substrate 110; and a second sample well 120 arranged in a second position on the substrate 110. In this example, the first sample well 120 can be configured to receive a first user sample—derived from a first human user—and a first fluorescent probe—configured to bind to a first target bioparticle—for detection of the first target bioparticle in the first user sample. Further, the second sample well 120 can be configured to receive a second user sample—derived from a second human user—and the first fluorescent probe for detection of the first target bioparticle in the second user sample. Therefore, the system 100 can be configured to test for presence of the first target bioparticle in samples collected from different users in a single cartridge 102. Additionally and/or alternatively, in the preceding example, the cartridge 102 can include: a third sample well 120 arranged in a third position on the substrate 110 and configured to receive the first user sample (e.g., a second aliquot of the first user sample)—derived from the first human user—and the second fluorescent probe—configured to bind to the second target bioparticle—for detection of the second target bioparticle in the first user sample. Therefore, the system 100 can be configured to test for presence of the first target bioparticle and the second target bioparticle in a single user sample collected from one user.

Further, in another implementation, the system 100 can be configured to autonomously process samples within the cartridge 102; and characterize presence of a set of target bioparticles within these samples based on detection of optical signals generated by fluorescent probes present in the set of samples. In particular, in this implementation, the system 100 can be configured to automatically process a cartridge 102—such as including an array of sample wells 120—for detection of a set of target bioparticles in user samples loaded onto the cartridge 102, such as by automatically: loading sample wells with samples and/or reagents assigned to these detection samples via a set of fluid dispensers 180; drain fluid and particulate from the set of sample wells 120 via loading of the cartridge 102 onto an automated draining system 100 configured to pump fluid from the sample well 120 and through the filter membrane 130; and coordinate motion of the reader 150 (e.g., an XYZ plane and/or in a Z plane) and/or the cartridge 102 (e.g., in an XY plane) in preparation for scanning of a particular sample well 120, in the set of sample wells 120, for presence of one or more target bioparticles.

By implementing autonomous processing of samples within the cartridge 102, the system 100 can thus minimize instances of false-positive and/or false-negatives due to human error and increase throughput by: increasing a quantity of sample wells on the substrate 110, increasing a rate of cartridge 102 processing by minimizing fluctuations in processing speed, and reducing downtime due to human error (e.g., loading of a reagent or sample into an incorrect sample well 120).

### 4. Sample Processing & Bioparticle Detection

The system 100 can include: a cartridge 102 configured to retain a target complex—formed via binding of a fluorescent probe to a target bioparticle—on a filter membrane 130 arranged within a sample well 120 in the cartridge 102; and a reader 150 configured to scan the sample well 120—including the filter membrane 130—for presence of the target complex.

In particular, the cartridge 102 can include: a substrate 110; a sample well 120 arranged on the substrate 110 and defining an upper opening 122 (or “sample well inlet”)—arranged on an upper surface 112 of the substrate 110—and a lower opening 124 (or “sample well outlet”) arranged on a lower surface 114 of the substrate 110; and a filter membrane 130 coupled to the substrate 110 and arranged across the lower surface 114 of the substrate 110, forming a barrier extending over the sample well outlet. The filter membrane 130 can define: an inner surface 132—facing the sample well 120—configured to contact fluid and particulate within the sample well 120; an outer surface 134; and a network of pores 136—extending between the inner surface 132 and the outer surface 134—and configured to convey fluid and biological particulate from the sample well 120 through the filter membrane 130 and prevent passage of the target complex through the filter membrane 130.

The reader 150 can include: a housing 192; a cartridge receptacle 160 configured to receive the cartridge 102 and locate the cartridge 102 the sample well 120 within a detection region within the housing 192; and a detector 152 defining a field of view intersecting the detection region and configured to detect an optical signal (e.g., a fluorescence signal) generated by contents of the sample well 120 (e.g., responsive to excitation of contents of the sample well 120).

Generally, in one implementation, to characterize presence of a target bioparticle (e.g., an antigen)—such as a virus, a biomarker (e.g., a tumor biomarker), a protein, etc.—present in a user sample derived from a human user (or “patient”)—such as a saliva sample, a blood sample, a serum sample, a urine sample, etc.—the user sample can initially be mixed with a probe solution to form a test solution. In particular, the probe solution can include a fluorescent probe—including a fluorescent molecule linked to a particular antibody corresponding to the target bioparticle—configured to selectively bind (e.g., with high specificity) to the target bioparticle to form a target complex. Therefore, if the user sample contains the target bioparticle, the fluorescent probe can bind to the target bioparticle—upon mixing of the user sample with probe solution—to form the target complex in the resulting test solution. Alternatively, if the user sample excludes the target bioparticle, the fluorescent probe will remain unbound within the resulting test solution.

In one example, in response to an amount (e.g., a quantity, a concentration) of the target bioparticle in the user sample falling below an amount of the fluorescent probe in the probe solution, a proportion of the amount of the fluorescent probe can bind to the amount of the target bioparticle to generate a first amount of the target complex. The resulting test solution—formed via mixing of the probe solution with the user sample—can therefore include: the first amount of the target complex in solution; and a second amount (or “remaining amount”) of the fluorescent probe—unbound to the target bioparticle—in solution, the second amount corresponding to a difference between the amount of the fluorescent probe and the proportion of the amount of the fluorescent probe bound to the target bioparticle.

In this implementation, the test solution can be added to the sample well 120 via the sample well inlet (e.g., via manual and/or automated pipetting) during a sample loading period. The absorbent pad stack 140 can be coupled to the outer surface 134 of the filter membrane 130 throughout the loading period to promote flow of fluid and biological particulate from the inner surface 132 and through pores 136 of the filter membrane 130. In particular, during the sample loading period: the sample well 120 can receive a volume (e.g., 0.5 milliliters) of the test solution; and the absorbent pad stack 140—coupled to the outer surface 134 of the filter membrane 130 throughout the loading period—can cooperate with the filter membrane 130 to draw fluid and biological particulate from the inner surface 132, through the network of pores 136, and onto the absorbent pad stack 140. The filter membrane 130—including the network of pores 136 of pore sizes within a target pore size range—can thus: drain fluid and biological particulate exhibiting sizes less than pore sizes within the target pore size range—such as including any remaining fluorescent probe unbound to the target bioparticle—from within the detection wall into the absorbent pad stack 140; and retain the target complex (e.g., each instance of the target complex)—configured to exhibit a target size exceeding pore sizes within the target pore size range—on the inner surface 132 of the filter membrane 130 and/or within the sample well 120.

Then, during a wash period, a volume of a wash buffer (e.g., 0.5 milliliters) can be added to the sample well 120 to rinse surfaces (e.g., walls, the inner surface 132 of the filter membrane 130) of the sample well 120 and remove any remaining particulate—other than the target complex—from the sample well 120 via draining of the wash buffer through the filter membrane 130. Upon completion of the wash period: the absorbent pad stack 140 can be removed from the cartridge 102; and the cartridge 102 can be inserted into the cartridge receptacle 160 within the reader 150. Then, a volume of a read buffer is added to the sample well 120 to promote suspension of the target complex—if present on the inner surface 132 of the sample membrane—within and/or across the sample well 120 prior to initiation of a detection period. The filter membrane 130—decoupled from the absorbent pad stack 140—enables retention of the volume of the read buffer within the sample well 120.

Finally, during the detection period, the detector 152 captures an optical signal—generated by fluid within the sample well 120—representing presence of the target bioparticle in fluid within the sample well 120 and thereby in the user sample. More specifically, during the detection period: an excitation source 154 (e.g., an array of LED lights), arranged proximal the cartridge receptacle 160, uniformly illuminates a detection region—containing the sample well 120—according to a target excitation intensity; the detector 152—defining a field of view intersecting the detection region—captures an optical signal (e.g., a fluorescence signal) emitted from the detection region and representing presence of the target bioparticle in the sample well 120. In particular, in one implementation, a controller 190 reads the optical signal captured by the detector 152; and interprets presence—such as binary presence (e.g., present or not present) and/or a magnitude of presence (e.g., an amount of the target bioparticle present)—of the target bioparticle based on the optical signal.

### 4.1 Variation: One Sample Well+Multiple Target Bioparticles

In one variation, the system 100 can be configured to detect presence of multiple target bioparticles in a single sample well 120. In particular, in this variation, the probe solution can be mixed to include: a first probe configured to bind to a first target bioparticle in a set of target bioparticles; and a second probe configured to bind to a second target bioparticle in the set of target bioparticles. This probe solution can then be mixed with a user sample to generate the test solution, thereby promoting formation of a first target complex—including the first target bioparticle bound to the first probe—and a second target complex—including the second target bioparticle bound to the second probe—based on presence of the first and second bioparticles in the user sample.

The test solution can then be processed on the cartridge 102 accordingly to: retain instances of the first target complex and instances of the second target complex on the inner surface 132 of the filter membrane 130 within the sample well 120; and release fluid and other biological particulate from the sample well 120 via the network of pores 136 of the filter membrane 130. The cartridge 102 can then be inserted into the reader 150 for detection of both: a first optical signal—representing presence of the first target bioparticle—based on presence of the first fluorescent probe in fluid in the sample well 120; and a second optical signal—representing presence of the second target bioparticle—based on presence of the second fluorescent probe in fluid in the sample well 120.

Additionally, in this variation, the probe solution can be mixed to further include: a third probe configured to bind to a third target bioparticle, in the set of target bioparticles, to form a third target complex; a fourth probe configured to bind to a fourth target bioparticle, in the set of target bioparticles, to form a fourth target complex; etc. The reader 150 can similarly be configured to detect: a third optical signal—representing presence of the third target bioparticle—based on presence of the third fluorescent probe in fluid in the sample well 120; and a fourth optical signal—representing presence of the fourth target bioparticle—based on presence of the fourth fluorescent probe in fluid in the sample well 120; etc.

### 5. Cartridge

The system 100 includes a cartridge 102 configured to insert into a reader 150 for detection of a target bioparticle in fluid contained in a sample well 120 within the cartridge 102.

In particular, the cartridge 102 can include a substrate 110: defining an upper surface 112 and a lower surface 114 (e.g., opposite and vertically below the upper surface 112 in an upright position); and a sample well 120—extending between an upper opening 122 (or a “well inlet”) arranged on the upper surface 112 and a lower opening 124 (or a “well outlet”) arranged on the lower surface 114—configured to receive a volume of a test solution via the upper opening 122, the test solution including a user sample mixed with a probe solution containing an amount of a fluorescent probe configured to bind with a target bioparticle to form a target complex. The cartridge 102 further includes a filter membrane 130 coupled to the lower surface 114 of the substrate 110 and extending across the lower opening 124, thereby forming a barrier at the well outlet. In particular, the filter membrane 130 can define: an inner surface 132 facing the sample well 120 and configured to receive fluid and particulate from the sample well 120; an outer surface 134 (e.g., opposite the inner surface 132); and a network of pores 136—extending between the inner surface 132 and the outer surface 134—configured to convey fluid and biological particulate from the sample well 120 through the filter membrane 130 and prevent passage of the target complex through the filter membrane 130.

The system 100 can further include a removable absorbent pad stack 140 (e.g., one or more absorbent pads)—removably coupled to the cartridge 102—configured to extend across the outer surface 134 of the filter membrane 130 when coupled to the cartridge 102. In particular, the absorbent pad stack 140 can be configured to cooperate with the filter membrane 130 to draw fluid from the inner surface 132, through the network of pores 136, and onto the absorbent pad stack 140.

The sample well 120, the filter membrane 130, and the absorbent pad stack 140 can therefore cooperate to: retain the target complex (e.g., one or more instances of the target complex)—formed of the fluorescent probe bound to the target bioparticle—on the inner surface 132 of the filter membrane 130 and/or within the sample well 120; and release fluid and other biological particulate—including any remaining fluorescent probe (or “unbound fluorescent probe”) unbound to the target bioparticle—through the network of pores 136 of the filter membrane 130 to the outer surface 134 and into the absorbent pad stack 140.

The cartridge 102 can be configured to insert into the reader 150 for detection of the target bioparticle in the test solution during a detection period. Upon completion of the detection period, the cartridge 102 can be removed from the reader 150 and replaced with a new cartridge 102 for detection of the target bioparticle—or a different target bioparticle—in a test solution contained in a sample well 120 of the new cartridge 102. Therefore, the system 100 can include a set of cartridge 102s, each cartridge 102, in the set of cartridge 102s, including a substrate 110, a sample well 120, and a filter membrane 130. Further, each cartridge 102, in the set of cartridge 102s, can be configured for disposal upon completion of each detection period. For example, the cartridge 102 can be formed of a disposable plastic material.

### 5.1 Sample Well

The cartridge 102 can include a sample well 120 integrated into the substrate 110 and configured to receive the test solution and other fluid for processing of the test solution.

In particular, the sample well 120 can be configured to receive: a volume of the test solution (e.g., 0.1 milliliters, 0.5 milliliters, 1 milliliter) including a user sample—derived from a human user—mixed with a probe solution during an initial loading period; a volume of a wash buffer (e.g., 0.1 milliliters, 0.5 milliliters, 1 milliliter)—configured to rinse the sample well 120 and convey fluid and biological particulate on surfaces of the sample well 120 toward the filter membrane 130—during a wash period succeeding the initial loading period; and a volume of a read buffer (e.g., 0.1 milliliters, 0.5 milliliters, 1 milliliter) during a final loading period succeeding the wash period. The sample well 120 can therefore be configured to exhibit a volume exceeding a maximum volume of fluid (e.g., 0.1 milliliters, 0.5 milliliters, 1 milliliter, 2 milliliters) contained in the sample well 120.

Further, the sample well 120 can define the upper opening 122 (or “well inlet”) arranged on the upper surface 112 of the substrate 110 and the lower opening 124 (or “well outlet”) arranged on the lower surface 114 of the substrate 110. In particular, the sample well 120 can be configured to define the upper opening 122 of a first area (e.g., cross-sectional area) and the lower opening 124 of a second area (e.g., cross-sectional area) greater than the first area, such that walls of the sample well 120—and the inner surface 132 of the filter membrane 130—are within a field of view of a detector 152 arranged vertically above the sample well 120 when the cartridge 102 is inserted in the reader 150 during the detection period. Further, by including walls of that taper inward from the upper surface 112 of the substrate 110 toward the lower surface 114 of the substrate 110, the sample well 120 enables spreading of the read buffer across a wider area—thereby improving detectability of an optical signal emitted by fluorescent probes suspended in the read buffer—while maintaining a relatively low cartridge 102 height.

### 5.1.1 Variation: Pre-Loaded Lyophilized Probe

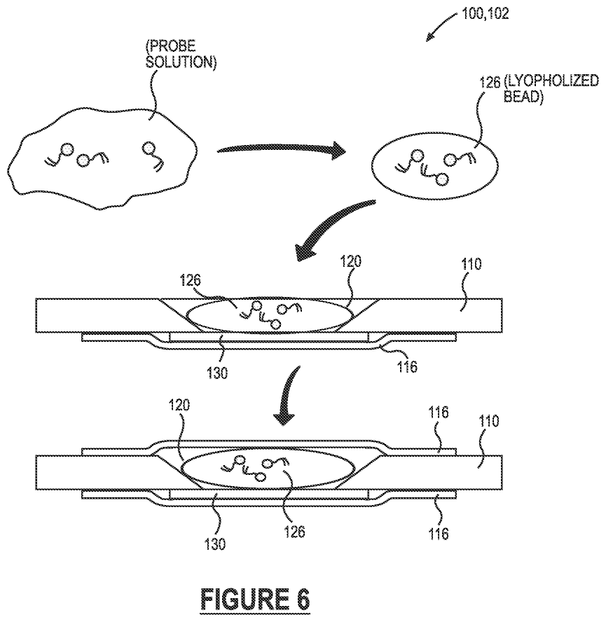

In one variation, as shown in FIGS. 6A and 6B, the cartridge 102 can be pre-loaded with a lyophilized probe configured to bind with a target bioparticle upon reconstitution of the lyophilized probe. In this variation, the user sample (e.g., saliva, blood, serum, or urine) can be directly added to the sample well 120—thereby reconstituting the lyophilized probe—for mixing with the probe solution.

In particular, in this variation, the cartridge 102 can include a probe layer 126 including lyophilized probes—each probe (e.g., fluorescent probe) including a fluorescent tag linked to a particular antibody configured to bind to the target bioparticle—arranged within the sample well 120. Additionally, in this variation, the cartridge can include a sealant 116 (e.g., a sealant tape)—removably coupled to the outer surface 134 of the filter membrane 130—configured to prevent flow of fluid and/or particulate through the filter membrane 130; and a sample well 120 cover—removably coupled to the upper surface 112 of the substrate 110—configured to cover the upper opening 122.

For example, the sealant 116 can initially be coupled to the outer surface 134 of the filter membrane 130, thereby preventing fluid from flowing through the filter membrane 130 and out of the cartridge 102. Then, the (liquid) probe solution can be added to the sample well 120. The probe solution can then be lyophilized within the sample well 120 to form a probe layer 126 of lyophilized probes (e.g., dried fluorescent probes) arranged within the sample well 120. The sample well 120 cover can then be attached to the upper surface 112 of the substrate 110 to prevent contamination of the sample well 120 and/or unintentional removal of the probe layer 126 from the sample well 120, such as during storage or transfer. Prior to addition of the user sample to the sample well 120, the sample well 120 cover can be removed from the upper surface 112 of the substrate 110. The user sample can then be added to the sample well 120 to: reconstitute the probe layer 126, thereby reforming the (liquid) probe solution; and mix with the probe solution to generate the test solution.

The sample well 120 cover can then be reattached to the upper surface 112 for a duration of a reaction period to promote complete mixing of the user sample and the probe solution and formation of the target complex. Finally, upon expiration of the reaction period, the sample well 120 cover can be removed from the upper surface 112 of the substrate 110 and the sealant 116 can be removed from the outer surface 134 of the filter membrane 130, in preparation for addition of the wash and/or read buffer to the sample well 120.

### 5.2 Filter Membrane

The cartridge 102 can include a filter membrane 130 coupled to the lower surface 114 of the substrate 110 and extending across the lower opening 124 of the sample well 120. In particular, the filter membrane 130 can define: an inner surface 132 facing the sample well 120 and configured to received fluid and particulate loaded in the sample well 120; an outer surface 134; and a network of pores 136 extending between the inner surface 132 and the outer surface 134.

The filter membrane 130 can be configured to: retain a target complex—formed of the fluorescent probe bound to the target bioparticle—on the inner surface 132 of the filter membrane 130 and/or within the sample well 120; and cooperate with an absorbent pad stack 140—transiently coupled to the outer surface 134 of the filter membrane 130—to convey fluid and biological particulate (e.g., excluding the target complex) through the network of pores 136 and into the absorbent pad stack 140. Further, when the absorbent pad is removed from the filter membrane 130—such as prior to addition of the read buffer to the sample well 120—the filter membrane 130 can be configured to prevent leaking of fluid from the sample well 120 and thereby retain fluid within the sample well 120.

In one implementation, the filter membrane 130 can exhibit a first size (e.g., a cross-sectional area, a diameter) greater than a second size of the lower opening 124 of the sample well 120, such that the filter membrane 130 extends across the second area of the lower opening 124, thereby minimizing sample loss. For example, the lower opening 124 can exhibit a diameter of 15 millimeters and the filter membrane 130 can exhibit a diameter between 15 millimeters and 20 millimeters.

In one implementation, the filter membrane 130 is attached to the substrate 110 via an adhesive layer. For example, the filter membrane 130 can be attached to the substrate 110 via an adhesive layer (e.g., adhesive tape) defining a first side—configured to adhere to surfaces of the filter membrane 130—and a second side—configured to adhere to surfaces of the substrate 110.

### 5.2.1 Network of Pores

The filter membrane 130 defines a network of pores 136: extending between the inner surface 132 and outer surfaces 134 of the filter membrane 130; and configured to convey fluid and biological particulate from the sample well 120 through the filter membrane 130 and prevent passage of the target complex through the filter membrane 130.

In one implementation, pores 136, in the network of pores 136, are configured to exhibit pore sizes within a target pore size range, such that the filter membrane 130 can: convey particles (e.g., in fluid), exhibiting sizes within and/or less than the target pore size range, through the network of pores 136 for removal from the sample well 120; and retain particles exhibiting sizes exceeding the target pore size range on the inner surface 132 of the filter membrane 130 and/or within the sample well 120.

In particular, in this implementation, the sample well 120 can be configured to receive the test solution—including the user sample mixed with an amount of the fluorescent probe—including a fluorescent tag bound to a target antibody configured to bind to the target bioparticle—and configured to bind with the target bioparticle to form the target complex, the fluorescent probe of a first size less than a second size of the target complex. The network of pores 136 of the filter membrane 130 can include pores 136 exhibiting sizes within the target pore size range, sizes within the target pore size range exceeding the first size of the fluorescent probe and falling below the second size of the target complex. Therefore, the filter membrane 130—in cooperation with the absorbent pad stack 140—can be configured to: release fluid including unbound fluorescent probe and/or other biological particulate from the sample well 120 and into the absorbent pad stack 140 via the network of pores 136; and retain the target complex on the inner surface 132 of the filter membrane 130 and/or within the sample membrane.

The system 100 can include a particular filter membrane 130 corresponding to a particular target bioparticle, such that the filter membrane 130 is configured to retain a target complex—formed of the target bioparticle bound to the fluorescent probe (e.g., a fluorescent tag linked to an antibody corresponding to the target bioparticle)—on the inner surface 132 of the filter membrane 130 and/or within the sample well 120, such as based on pore size of pores in the network of pores 136 of the filter membrane 130. For example, a first instance of the system 100—configured to detect presence of a bacteria—can include a first filter membrane 130 including pores within a first pore size range corresponding to a first size of a first target complex formed of the bacteria bound to the fluorescent probe. A second instance of the system 100—configured to detect presence of a protein (e.g., a biomarker)—can include a second filter membrane 130 including pores within a second pore size range corresponding to a second size of a second target complex formed of the protein bound to the fluorescent probe. Finally, a third instance of the system 100—configured to detect presence of a virus—can include a third filter membrane 130 including pores within a third pore size range corresponding to a third size of a third target complex formed of the virus bound to the fluorescent probe.

The filter membrane 130 can therefore be selected based on a size of the target complex formed of the target bioparticle bound to the fluorescent probe. For example, the system 100 can include a filter membrane 130 including a network of pores 136 exhibiting pore sizes of approximately (e.g., within 5 percent, within 10 percent) 0.20 microns. In this example, the filter membrane 130 can be configured to: retain biological particulate of sizes exceeding 0.20 microns on the inner surface 132 of the filter membrane 130 and/or within the sample well 120; and drain (e.g., release, convey) fluid and biological particulate of sizes less than 0.20 microns through the network of pores 136—toward the outer surface 134 of the filter membrane 130—and onto the absorbent pad 140 coupled to the outer surface 134. Therefore, the system 100 can be configured to retain instances of a target complex—including a bioparticle bound to a fluorescent probe and exhibiting a size greater than 0.20 microns—on the inner face 132 of the filter membrane 130 and/or within the sample well 130, thereby enabling detection of the target bioparticle.

Additionally, in this implementation, the filter membrane can be selected based on a size of the target complex—formed of the target bioparticle bound to the fluorescent probe—and a threshold drain rate for releasing fluid and other biological particulate from the sample well. In particular, the filter membrane can be configured to include a network of pores exhibiting sizes within a target pore size range, pore sizes within the target pore size: less than a size of the target complex; and greater than a threshold pore size corresponding to a threshold drain rate. The filter membrane 130 can therefore be configured to: minimize and/or restrict passage of the target complex through the network of pores 136, thereby maximizing a probability of detection of the target bioparticle in a sample loaded in the sample well 130; and maximize a drain rate of the filter membrane 130—or a flowrate of fluid through the filter membrane 130—thereby increasing throughput of the system 100 by reducing drain time thus minimizing latency between loading of the test solution into the sample well 120 and final detection of the target complex in the sample well 120 via the reader 150. Further, by maximizing a pore size of pores in the network of pores 136—while maintaining the pore size below a size of the target complex—the system 100 can maximize draining of other biological particulate—excluding the target bioparticle—present in fluid in the sample well 120. By maximizing removal of other biological particulate from the sample well 120, the system 100 can therefore increase concentration of the target complex of the inner surface 132 of the filter membrane 130 and/or within the sample well 120, thereby increasing strength of a signal generated by the fluorescent probe of the target complex during detection.

Alternatively, in one variation, described further below, in which the cartridge 102 includes a capture-antibody layer 127 arranged within the sample well 120 and/or on the inner face of the filter membrane 130, the network of pores 136 can be configured to include pores 136 within a relatively-higher pore size range. In particular, because the capture-antibody layer 127 retains the target bioparticle within the sample well 120, pores 136 of the network of pores 136 can be configured to enable flow of larger particles through the network of pores 136, thereby enabling inclusion of pores 136 of greater sizes. For example, in this variation, pores 136, in the network of pores 136, can be configured to exhibit sizes within a target pore size range, sizes within the target pore size range greater than a size of the fluorescent probe and greater than size of the target complex.

### 5.3 Variation: Multiple Sample Wells

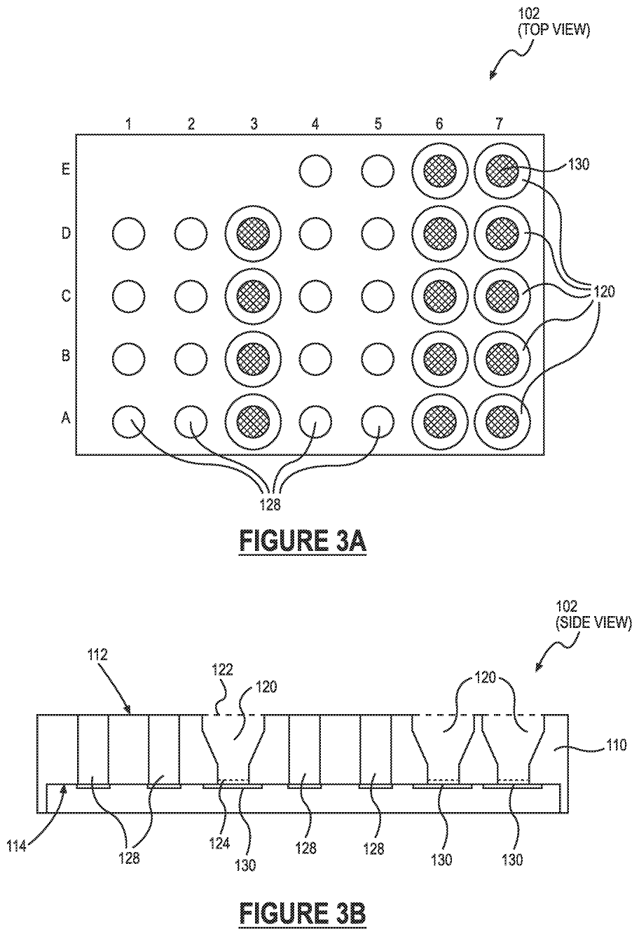

In one variation, as shown in FIGS. 3A and 3B, the substrate 110 can include multiple sample wells 120 (e.g., 2 sample wells 120, 10 sample wells 120, 30 sample wells 120, 100 sample wells 120) arranged on the substrate 110. In particular, in this variation, the cartridge 102 can include: a substrate 110; a set of sample wells 120 integrated within the substrate 110, each sample well 120, in the set of sample wells 120, extending between the upper surface 112 of the substrate 110 and the lower surface 114 of the substrate 110 and configured to receive a test solution formed via mixing of a user sample with a probe solution. In this variation, sample wells 120 can be arranged on the substrate 110 such that each sample well 120 is arranged at least a minimum distance from each neighboring sample well 120, thereby limiting contamination between sample wells 120.

For example, the cartridge 102 can include: a first sample well 120—extending between a first upper opening 122 arranged on the upper surface 112 and a first lower opening 124 arranged on the lower surface 114—configured to receive a first test solution; and a second sample well 120—extending between a second upper opening 122 arranged on the upper surface 112 and a second lower opening 124 arranged on the lower surface 114—configured to receive a second test solution. In this example, the first sample well 120 can define a first central axis parallel and offset (e.g., a by a threshold distance) a second central axis of the second sample well 120, wherein both the first central axis and the second central axis are normal the upper surface 112 and the lower surface 114.

In the preceding example, the cartridge 102 can include: a first filter membrane 130 coupled to the lower surface 114 (e.g., a first region of the lower surface 114) of the substrate 110 and extending across the first lower opening 124; and a second filter membrane 130 coupled to the lower surface 114 (e.g., a second region of the lower surface 114) of the substrate 110 and extending across the second lower opening 124.

In one implementation, each sample well 120, in the set of sample wells 120, can be assigned to a particular human user in a set of human users. In particular, in this implementation, each sample well 120, in the set of sample wells 120, can be configured to receive a unique test solution including a particular user sample, from a set of user samples, mixed with a probe solution including a fluorescent probe configured to bind to a target bioparticle. For example, the cartridge 102 can include: a first sample well 120—extending between a first upper opening 122 arranged on the upper surface 112 and a first lower opening 124 arranged on the lower surface 114—configured to receive a first test solution including a first user sample, derived from a first human user, mixed with a fluorescent probe in solution, the fluorescent probe configured to bind to a target bioparticle; and a second sample well 120—extending between a second upper opening 122 arranged on the upper surface 112 and a second lower opening 124 arranged on the lower surface 114—configured to receive a second test solution including a second user sample, derived from a second human user distinct from the first human user, mixed with the fluorescent probe in solution.

Additionally and/or alternatively, in another implementation, each sample well 120, in the set of sample wells 120, can be assigned to a particular target bioparticle in a set of target bioparticles. In particular, in this implementation, each sample well 120, in the set of sample wells 120, can be configured to receive a unique test solution including a subvolume of user sample mixed with a fluorescent probe, in a set of fluorescent probes, in solution, the fluorescent probe configured to bind to a target bioparticle in the set of target bioparticles. For example, the cartridge 102 can include: a first sample well 120—extending between a first upper opening 122 arranged on the upper surface 112 and a first lower opening 124 arranged on the lower surface 114—configured to receive a first subvolume of a test solution, derived from a human user, mixed with a first fluorescent probe in solution, the first fluorescent probe configured to bind to a first target bioparticle in a set of target bioparticles; and a second sample well 120—extending between a second upper opening 122 arranged on the upper surface 112 and a second lower opening 124 arranged on the lower surface 114—configured to receive a second test solution including a second subvolume of the user sample mixed with a second fluorescent probe in solution, the second fluorescent probe configured to bind to a second target bioparticle, in the set of target bioparticles.

Additionally and/or alternatively, in another implementation, the cartridge 102 can be configured to include a set of sample wells 120 including: a first subset of sample wells 120 configured to receive a first user sample derived from a first human user; and a second subset of sample wells 120 configured to receive a second user sample derived from a second human user. In this implementation, the first subset of sample wells 120 can include: a first sample well 120 configured to receive a first test solution including a first subvolume of the first user sample, derived from the first human user, mixed with a first fluorescent probe in solution, the first fluorescent probe configured to bind to a first target bioparticle; and a second sample well 120 configured to receive a second test solution including a second subvolume of the first user sample mixed with a second fluorescent probe in solution, the second fluorescent probe configured to bind to a second target bioparticle. The second subset of sample wells 120 can similarly include: a third sample well 120 configured to receive a third test solution including a first subvolume of the second user sample, derived from the second human user, mixed with the first fluorescent probe in solution; and a fourth sample well 120 configured to receive a fourth test solution including a second subvolume of the second user sample mixed with the second fluorescent probe in solution. In this example, the system 100 can therefore enable testing for presence of both a first and second target bioparticle—in sample collected for both a first and second human user—within a single cartridge 102.

In one variation, the substrate 110 can include a set of reagent wells 128—distinct from the set of sample wells 120—configured to store a set of reagents, such as a wash buffer and/or a read buffer, configured to be added to sample well 120 for mixing with a user sample and/or test solution. In this variation, the substrate 110 can include a set of fluidic channels 118 configured to transfer reagents from the set of reagent wells 128 into the set of sample wells 120. Alternatively, the set of reagents can be manually and/or autonomously transferred.

### 5.3.1 Example: Virus

In one implementation, the system 100 can be configured to detect presence of a virus in a user sample (e.g., derived from a human user).

For example, the system 100 can be configured to detect presence of coronavirus in a first saliva sample collected from a first human user. In this example, a first volume of the saliva sample can be mixed with a first volume of a probe solution—including an amount of a first fluorescent probe configured to bind to a first coronavirus antigen, such as linked to a first variant of coronavirus (e.g., delta variant), to form a first target complex—to generate a first test solution. A volume of the first test solution can then be added to a first sample well 120, in a set of sample wells 120, on the substrate 110 for further processing on the cartridge 102 and detection of the.

Additionally, in this example, a second volume of the first saliva sample can be mixed with a volume of a second probe solution—including an amount of a second fluorescent probe configured to bind to a second coronavirus antigen, such as linked to a second variant of coronavirus (e.g., omicron variant)—to generate a second test solution. A volume of the second test solution can then be added to a second sample well 120, in the set of sample wells 120, on the substrate 110 for further processing. Additional volumes of the first saliva sample can similarly be mixed with volumes of additional probe solutions—corresponding to different variants of coronavirus—to generate additional test solutions from this singular saliva sample collected from the first human user. Each test solution can be added to a particular sample well 120, in the set of sample wells 120, on the substrate 110 of the cartridge 102.

Additionally and/or alternatively, in this example, a volume of a second saliva sample—derived from a second user—can be mixed with a volume of the first probe solution—including an amount of the first fluorescent probe configured to bind to the first coronavirus antigen—to generate a third test solution. A volume of the third test solution can then be added to a third sample well 120, in the set of sample wells 120, on the substrate 110 for further processing. Additional volumes of the second saliva sample can similarly be mixed with volumes of additional probe solutions—corresponding to different variants of coronavirus—to generate additional test solutions from this singular saliva sample collected from the second human user.

Therefore, in this implementation, the cartridge 102 can be configured to include a set of sample wells 120 including: a first subset of sample wells 120 assigned to testing for presence of a set of virus antigens—each virus antigen, in the set of virus antigens, assigned to a particular sample well 120 in the first subset of sample wells 120—in a first user sample (e.g., a first saliva sample) derived from a first user (e.g., a first human patient); a second subset of sample wells 120 assigned to testing for presence of the set of virus antigens—each virus antigen, in the set of virus antigens, assigned to a particular sample well 120 in the second subset of sample wells 120—in a second user sample (e.g., a second saliva sample) derived from a second user (e.g., a second human patient); a third subset of sample wells 120 assigned to testing for presence of the set of virus antigens—each virus antigen, in the set of virus antigens, assigned to a particular sample well 120 in the third subset of sample wells 120—in a third user sample (e.g., a third saliva sample) derived from a third user (e.g., a third human patient); etc.

### 5.3.2 Cancer Biomarkers

In one implementation, the system 100 can be configured to detect presence of a set of biomarkers—linked to a particular form of cancer—in a user sample.

For example, the cartridge 102 and reader 150 can cooperate to detect presence of a set of biomarkers linked to ovarian cancer. In this example, the cartridge 102 can include a first set of sample wells 120 arranged on the substrate 110 and including: a first sample well 120 configured to receive a first test sample—including a first subvolume of the first plasma sample, derived from a first human user, mixed with a first fluorescent probe configured to bind to a first biomarker to form a first target complex; a second sample well 120 configured to receive a second test sample—including a second subvolume of the first plasma sample mixed with a second fluorescent probe configured to bind to a second biomarker to form a second target complex; and a third sample well 120 configured to receive a second test sample—including a third subvolume of the first plasma sample mixed with a third fluorescent probe configured to bind to a third biomarker to form a third target complex.

In particular, a first serum sample can be extracted from a blood sample provided by a first patient. Then, during a sample preparation period: the first subvolume of the serum sample can be mixed with a volume of a first probe solution—including an amount of the first fluorescent probe—to generate the first test sample; the second subvolume of the serum sample can be mixed with a volume of a second probe solution—including an amount of the second fluorescent probe—to generate the second test sample; and the third subvolume of the serum sample can be mixed with a volume of a third probe solution—including an amount of the third fluorescent probe—to generate the third test sample.

Then, during a loading period succeeding the sample preparation period: the first test sample can be added to the first sample well 120; the second test sample can be added to the second sample well 120; the third test sample can be added to the third sample well 120; and fluid and biological particulate in each sample well 120, in the set of sample wells 120, can be drained out of the sample wells 120 through a filter membrane 130, in a set of filter membrane 130s, attached to the lower opening 124 of the sample well 120. During a wash period succeeding the loading period, a volume of a wash buffer can be added to each sample well 120 in the set of sample wells 120 and drained out of the sample well 120 through the filter membrane 130. Finally, the absorbent pad stack 140 can be removed from the cartridge 102 and the cartridge 102 can be inserted into the read in preparation for the detection period. A volume of a read buffer can then be added to each sample well 120 in the set of sample wells 120.

Then, during the detection period, the photodetector can: detect a first fluorescence signal—such as at a particular wavelength and/or intensity—generated by fluid within the first sample well 120 and representing an amount of the first biomarker in the first sample well 120; detect a second fluorescence signal generated by fluid within the second sample well 120 and representing an amount of the second biomarker in the second sample well 120; and detect a third fluorescence signal generated by fluid within the third sample well 120 and representing an amount of the third biomarker in the third sample well 120.

A computer system 100 configured to interface with the system 100 can then: access the amount of each biomarker detected; access a model linking amounts of biomarkers detected to a set of ovarian cancer diagnoses, such as a negative diagnosis, a positive diagnosis, a positive diagnosis at a particular stage (e.g., Stage I, II, III, or IV); and output a confidence score for a particular diagnosis based on the amounts of the first, second, and third biomarker and the model. Alternatively, a physician of the first patient can interpret a diagnosis for the first patient based on the amounts of the first, second, and third biomarker detected.

### 5.3.3 Allergen Testing

In one variation, the system 100 can be configured to detect presence of a set of immunoglobulin E antibodies (or “IGEs”)—linked to a set of allergens—in a user sample.

In this variation, the user sample (e.g., a serum sample) can be loaded into the sample well 120 prior to mixing with the probe solution. In particular, in this variation, the user sample—including an amount of a target IGE linked to a target allergen and an amount of secondary IGEs (e.g., linked to other allergens)—can be added to the sample well 120 at a first time during a setup period. Then, at a second time succeeding the first time within the setup period, an allergen solution—including an amount of a target allergen configured to selectively bind to the target IGE to form an allergen-IGE complex—can be added to the user sample within the sample well to generate an allergen-IGE solution. The allergen solution and the user sample can be held in the sample well 120 for an incubation period of a target duration (e.g., 10 minutes, 15 minutes, 30 minutes) to enable complete mixing and binding of each target IGE present in the user sample to a target allergen present in the allergen solution. Throughout the setup period and the incubation period, the absorbent pad stack 140 can be decoupled from the cartridge 102 to prevent fluid from flowing through filter membrane 130.

Then, upon completion of the incubation period, the absorbent pad stack 140 can be recoupled to the cartridge 102 to promote flow of fluid and biological particulate—including secondary IGEs and unbound target allergen and excluding the allergen-IGE complex—through the network of pores 136 in the filter membrane 130. Upon completion of draining of the sample well 120, the absorbent pad stack 140 can be removed from the substrate 110 in preparation for addition of the probe solution. Then, the probe solution—including an amount of a fluorescent probe including a fluorescent marker bound to a target anti-IGE—can be added to the sample well 120 for mixing with the allergen-IGE solution. Because the target IGE is the only IGE present in the sample well 120—after draining of the amount of secondary IGEs from the sample well 120 and into the absorbent pad stack 140—the fluorescent probe can bind to the target IGE, in the target allergen-IGE complex, to form an IGE-allergen-probe complex (i.e., the target complex). The absorbent pad stack 140 can then be recoupled to the cartridge 102 to promote flow of fluid and biological particulate—including unbound target anti-IGE and excluding the IGE-allergen-probe complex—through the network of pores 136 in the filter membrane 130.

The cartridge 102 can then be inserted into the reader 150 for detection of the IGE-allergen-probe complex. In particular, the reader 150 can detect an optical signal—(e.g., a fluorescence signal) generated by the fluorescent probe responsive to excitation of the fluorescent probe via activation of an excitation source 154 (e.g., a UV LED)—representing presence of the target IGE in the sample well 120 and thereby in the user sample. For example, the reader 150 can detect an optical signal of a particular magnitude—such as fluorescence at a particular wavelength and/or intensity—corresponding to an amount (e.g., quantity, concentration) of the target IGE in the user sample. The system 100 can therefore be configured to enable characterization of a user's sensitivity to a particular allergen based on the amount of the corresponding target IGE detected in the user sample.

In one example, the cartridge 102 and reader 150 can cooperate to detect presence of: a first IGE linked to a pollen allergen; a second IGE linked to a dandruff allergen; a third IGE linked to a food allergen (e.g., a dairy, nut, or gluten allergen); a fourth IGE linked to a medicinal allergen; a fifth IGE linked to a material allergen (e.g., a latex allergen); etc. The system 100 can thus process a user sample (e.g., a serum sample) according to the methods and techniques described above to detect presence of each of these IGEs in the user sample and therefore enable characterization of the user's sensitivity to each of the corresponding allergens.

### 5.4 Variation: PCR

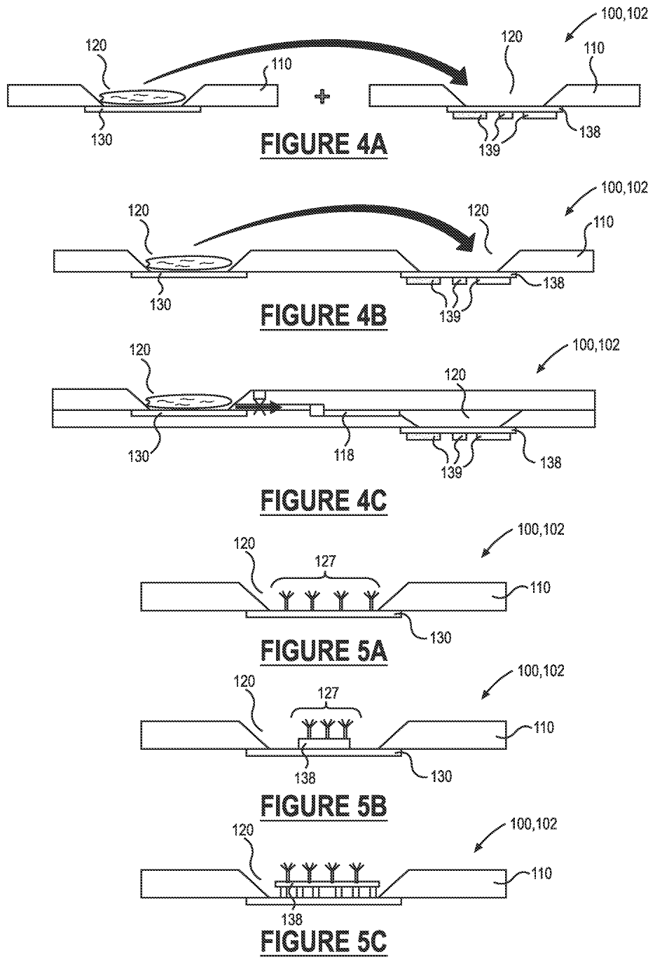

In one variation, as shown in FIGS. 4A-4C, the system 100 can be configured to enable detection of the target bioparticle via implementation of an amplification assay (e.g., a PCR amplification assay) in combination with implementation of the immunoassay described above.

In one implementation, the cartridge 102 can include a first sample well 120 configured for execution of the immunoassay and a second sample well 120 configured for execution of the amplification assay. In particular, in this implementation, the cartridge 102 can include: the first sample well 120—configured for execution of the immunoassay—arranged on the substrate 110 and extending between a first upper opening 122 arranged on the upper surface 112 and a first lower opening 124 arranged on the lower surface 114; a filter membrane 130 coupled to the lower surface 114 and extending across the first lower opening 124; the second sample well 120—fluidly coupled to the first sample well and configured for execution of the amplification assay—arranged on the substrate 110 and extending between a second upper opening 122 arranged on the upper surface 112 and a second lower opening 124 arranged on the lower surface 114; a base plate 138 (e.g., a metal base plate 138) coupled to the lower surface 114 of the substrate 110 and extending across the second lower opening 124; and a set of heating elements 139 (e.g., a Peltier device and/or heater element) arranged within the base plate 138 and configured to regulate a temperature of the base plate 138 according to an amplification assay (e.g., a PCR amplification assay).

In the preceding implementation, a test sample can be added to the first sample well 120 at a first time, which can be processed within the cartridge 102 according to the methods and techniques described above. Then, during a first detection period: the cartridge 102 can be inserted into the cartridge receptacle 160 of the reader 150 to locate the first sample well 120 within the detection region; the first sample well 120 can receive a volume of the read buffer to form a filtered test solution in the first sample well 120; and the photodetector can detect a first optical signal generated by the filtered test sample in the first sample well 120, the filtered test sample including an amount of a target complex—formed of the fluorescent probe bound to the target bioparticle (e.g., a target antigen)—in the read buffer.

Then, in response to completion of the first detection period, the second sample well 120 can receive the filtered test sample from the first sample well 120. For example, the substrate 110 can include: a fluidic channel 118 extending between the first and second sample well 120; and a valve arranged within the fluidic channel 118 and configured to transiently open to enable fluid flow from the first sample well 120 toward the second sample well 120.

Once the filtered test sample is loaded into the second sample well 120, contents of the second sample well 120 can be processed according to the amplification assay (e.g., a PCR amplification assay). For example, the cartridge 102 can further include: a temperature sensor coupled to the set of heating elements 139; and a controller 190 including a set of electronics and configured to operate the set of heating elements 139 according to the PCR amplification assay and based on a reading of the temperature sensor, thereby enabling temperature cycling of contents of the second reaction well for amplification of a set of target nucleic acid segments. Further, reagents for the amplification assay—such as corresponding to lysis, transcription and hybridization steps of a PCR amplification assay—can be added to the second sample well 120 according to the amplification assay. Therefore, the cartridge 102 can enable lysis, reverse transcription, hybridization of a target nucleic acid segments to a fluorescent probe—including a fluorescent tag and a quencher tag corresponding to a segment complimentary to the target nucleic acid segment. Upon amplification of the target nucleic acid segment, the quencher tag—bound to the segment complementary to the target nucleic acid segment—is released from the fluorescent probe.

In response to completion of the amplification assay, during a second detection period: the cartridge 102 can again be inserted into the cartridge receptacle 160 of the reader 150 to locate the second sample well 120 within the detection region of the photodetector; the second sample well 120 can receive an amount of an amplification buffer; and the photodetector can detect a second optical signal generated by fluid in the second sample well 120 and representing presence of the target bioparticle in fluid in the second sample well 120 during the second detection period.

In one implementation, as shown in FIG. 4C, the first sample well 120—configured for execution of the immunoassay—can be fluidly coupled to the second sample well 120—configured for execution of the amplification assay (e.g., a PCR amplification assay)—via a channel 118 extending between the first and second sample wells 120. The cartridge can therefore include: a fluidic channel 118—integrated into the substrate 110—extending between the first and second sample wells 120; and a valve 119—arranged within the fluidic channel—configured to rotate between a closed position and an open position to transiently disable and enable fluid flow from the first sample well 120, through the fluidic channel 118, and into the second sample well 120 in preparation for execution of the amplification assay in the second sample well 120. Alternatively, in another implementation, as shown in FIG. 4B, the cartridge can include the first sample well 120—configured for execution of the immunoassay—and the second sample well 120—configured for execution of the amplification assay—without a fluidic channel 118. In this implementation, fluid can be transferred—such as manually by a lab technician and/or by an automated fluid dispenser 180—from the first sample well 120 to the second sample well 120 by drawing fluid out of the first sample well 120 and releasing this fluid into the second sample well 120. Alternatively, in yet another implementation, as shown in FIG. 4A, the system 100 can include: a first cartridge including a first sample well—including the filter membrane 130—configured for execution of the immunoassay; and a second cartridge including a second sample well—including the base plate 138 and the set of heating elements 139—configured for execution of the amplification assay.

### 5.5 Variation: Capture Antibody

In one variation, as shown in FIGS. 5A-5C, the cartridge 102 can be pre-loaded with a capture antibody configured to bind to a particular target bioparticle. In particular, the cartridge 102 can be configured to include a capture antibody—immobilized on a surface of the filter membrane 130 and/or within the sample well 120—configured to bind to a target bioparticle present in a test solution added to the sample well 120. In this variation, the cartridge 102 can include a capture antibody that is highly-specific to the particular target bioparticle, such that the capture antibody selectively binds to the target bioparticle over other bioparticles (e.g., antigens) present in test solution, thereby maximizing detectability of the target bioparticle in the test solution.

In one implementation, the cartridge 102 can include a capture antibody—configured to bind to a particular target bioparticle—coupled directly to the filter membrane 130. For example, the cartridge 102 can include an amount (e.g., a quantity, a concentration, a proportion) of a capture antibody coupled directly to the inner surface 132 of the filter membrane 130.

Alternatively, in another implementation, the cartridge 102 can include: a base structure coupled to the filter membrane 130 and extending from the inner surface 132 of the filter membrane 130 into the sample well 120; and an amount of a capture antibody coupled to the base structure. For example, the cartridge 102 can include: a thin, antibody substrate 110 (e.g., a flat, rectangular or rounded substrate 110)—such as exhibiting less than a threshold thickness—attached to the inner surface 132 of the filter membrane 130 and extending into the sample well 120; and an amount of a capture antibody coupled to this antibody substrate 110. In this example, the antibody substrate 110 can be formed of a particular material—such as glass, silicon, plastic, etc.—and attached to the filter membrane 130 via gluing, heating, and/or ultrasonic welding of the antibody substrate 110 to the filter membrane 130. In another example, the cartridge 102 can include a grid structure coupled to the inner surface 132 of the filter membrane 130 and extending into the sample well 120; and an amount of a capture antibody coupled to the grid structure.

In this implementation, the base structure can be configured to exhibit an area (e.g., a cross-sectional area) less than an area of the filter membrane 130, such that a threshold proportion of the inner surface 132 of the filter membrane 130 is uncovered by the base structure, thereby enabling efficient washing of the filter membrane 130 prior to insertion of the cartridge 102 into the reader 150.

Alternatively, in another implementation, the cartridge 102 can include a capture antibody—configured to bind to a particular target bioparticle—coupled to walls of the sample well 120. In particular, in this implementation, the cartridge 102 can include: a base structure (e.g., antibody substrate 110, grid structure) arranged within the sample well 120 and attached to the walls (e.g., plastic walls) of the sample well 120; and an amount of a capture antibody coupled to the base structure accordingly.

In the preceding implementations, the base structure can be configured to exhibit an area (e.g., a cross-sectional area) less than an area of the filter membrane 130, such that a threshold proportion of the inner surface 132 of the filter membrane 130 is uncovered by the base structure, thereby enabling efficient (e.g., rapid, thorough) washing of the filter membrane 130 prior to insertion of the cartridge 102 into the reader 150. Upon insertion of the cartridge 102 into the reader 150, the cartridge receptacle 160 can thus locate the sample well 120—including the filter membrane 130 and the base structure forming a bottom surface of the sample well 120—within the detection region (e.g., defined by the field of view of photodetector).

In this variation, by leveraging a capture antibody to bind and retain the target bioparticle on the inner surface 132 of the filter membrane 130 and/or within the sample well 120—rather than relying on size-based filtering of particles via the network of pores 136 of the filter membrane 130—the filter membrane 130 can be configured to include pores 136 exhibiting sizes within a higher size range, thereby increasing throughput of the system 100 by increasing a rate of fluid flow through the filter membrane 130, such as during washing of the filter membrane 130 prior to insertion of the cartridge 102 into the reader 150. For example, the filter membrane 130 can define the network of pores 136—extending between the inner surface 132 and the outer surface 134 of the filter membrane 130—exhibiting pore sizes within a target pore size range exceeding a threshold pore size, such as exceeding a size of the target bioparticle and/or of the target complex formed of the target bioparticle bound to the probe.

Further, in this variation, the cartridge 102 can include a capture antibody that is distinct from an antibody forming the fluorescent probe and thereby present in the probe solution added to the sample well 120. By including a unique capture antibody that is distinct from the antibody forming the fluorescent probe—thereby enabling binding of the target bioparticle to both the capture antibody (e.g., at a first binding site defined for the capture antibody) and the antibody of the fluorescent probe (e.g., at a second binding site defined for the antibody)—the cartridge 102 can therefore maximize retention of the target bioparticle on the filter membrane 130 and/or within the sample well 120 and thus maximize detection of the target bioparticle.

### 6. Reader

The system 100 includes a reader 150 configured to receive the cartridge 102 for detection of the target bioparticle in fluid contained in the sample well 120.

In particular, the reader 150 can include: a housing 192; a cartridge receptacle 160 configured to transiently receive the cartridge 102 and locate the sample well 120 within a detection region within the housing 192; an excitation source 154 arranged proximal the cartridge receptacle 160 within the housing 192 and configured to illuminate the detection region according to a target excitation intensity; and a detector 152 arranged within the housing 192, defining a field of view intersecting the detection region, and configured to detect an optical signal (e.g., a fluorescence signal)—generated by fluid in the sample well 120 located within the detection region—representing presence of the target bioparticle in the sample well 120.

In one implementation, the detector 152 can include a photodetector (e.g., a single-pixel photodetector) configured to convert the optical signal—representing presence of the target bioparticle in the sample well 120—to an electrical signal. In this implementation, the reader 150 can further include an amplifier 158 coupled to the photodetector and configured to amplify the electrical signal output by the photodetector; and a controller 190 configured to read the electrical signal output by the amplifier 158 and interpret presence of the target bioparticle based on the electrical signal.

For example, the reader 150 can include: a light-tight enclosure—devoid of light—forming the housing 192; an array of LEDs arranged proximal the cartridge receptacle 160 within the housing 192 and configured to illuminate the detection region according to a target excitation intensity to elicit the optical signal; and a detector 152 arranged above the cartridge receptacle 160 within the housing 192 and configured to detect an optical signal emitted from the sample well 120, located in the detection region, responsive to activation of LEDs in the array of LEDs (e.g., responsive to illumination of the detection region).

Additionally and/or alternatively, in one variation, the reader 150 can further include an optical emission filter: arranged between the photodetector and the detection region; and configured to attenuate wavelengths outside of an emission wavelength range.

### 6.1 Variation: Multiple Target Bioparticles

In one variation, in which the sample well 120 is configured to receive a test sample including multiple fluorescent probes configured to bind to multiple target bioparticles, as described above, the reader 150 can be configured to detect multiple optical signals output by contents of the sample well 120 and therefore detect presence of multiple target bioparticles within the test sample.

In one example, the sample well 120 can initially receive a test solution including the user sample mixed with the probe solution including: a first amount of a first fluorescent probe configured to bind with a first target bioparticle to form a first target complex; and a second amount of a second fluorescent probe configured to bind with a second target bioparticle to form a second target complex. The test solution can then be processed—according to the methods and techniques described above—to retain the first and second target bioparticle within the sample well 120 and remove all other fluid and particulate from the sample well 120 via the network of pores 136 of the filter membrane 130. The cartridge 102 can then be loaded into the reader 150 for detection of the first and second target bioparticle. In particular, in this example, the photodetector is configured to: detect a first optical signal (e.g., a first fluorescence signal)—output by the first fluorescent probe in the first target complex—representing presence of the first target bioparticle in fluid in the sample well 120; and detect a second optical signal (e.g., a second fluorescence signal)—output by the second fluorescent probe in the second target complex—representing presence of the second target bioparticle in fluid in the sample well 120.

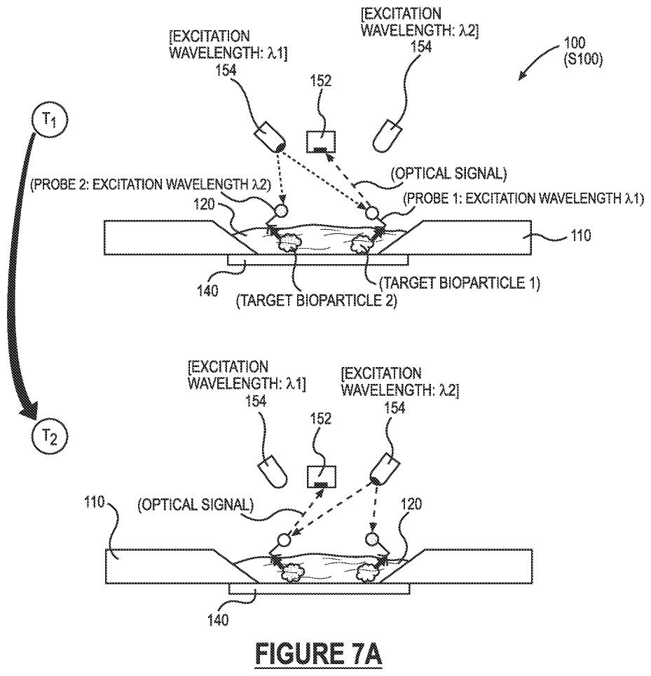

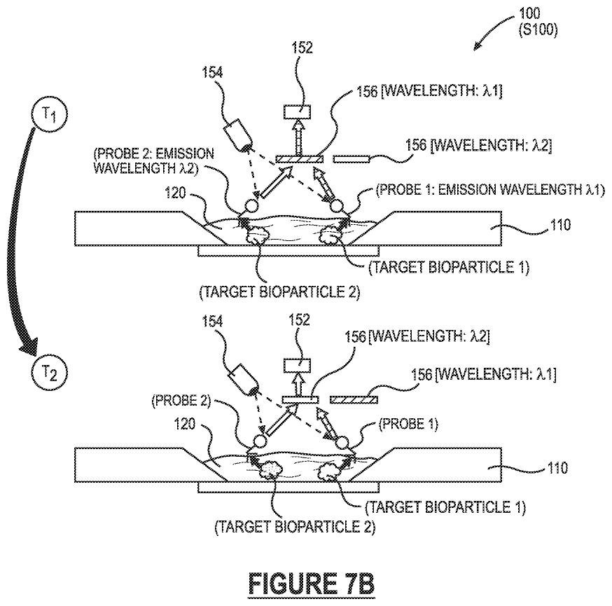

In one implementation, as shown in FIG. 7B, the reader 150 can be configured to include a set of optical filters, each filter, in the set of optical filters, corresponding to detection of a particular target bioparticle in a set of target bioparticles. For example, the sample well 120 can be configured to receive the test solution including the user sample mixed with the probe solution including: a first amount of a first fluorescent probe configured to bind with a first target bioparticle, in a set of target bioparticles, defining a first emission wavelength range; and a second amount of a second fluorescent probe configured to bind with a second target bioparticle, in a set of target bioparticles, defining a second emission wavelength range, wavelengths within the second emission wavelength range exceeding wavelengths within the first emission wavelength range. In this example, the reader 150 can include: a first optical filter—transiently arranged between the photodetector and the detection region—configured to block detection of wavelengths, by the photodetector, outside of the first emission wavelength range; and a second optical filter—transiently arranged between the photodetector and the detection region—configured to block detection of wavelengths, by the photodetector, outside of the second emission wavelength range. The photodetector can therefore be configured to: detect the first optical signal (e.g., fluorescence) in the first emission wavelength range, representing presence of the first target bioparticle in fluid in the sample well 120; and detect the second optical signal (e.g., fluorescence) in the second emission wavelength range, representing presence of the second target bioparticle in fluid in the sample well 120.

Alternatively, in another implementation, as shown in FIG. 7A, the excitation source 154 can be configured to illuminate the detection region at a particular excitation wavelength corresponding to a target excitation wavelength of the fluorescent probe. In particular, in this implementation, the sample well 120 can be configured to receive the test solution including the user sample mixed with the probe solution including: a first amount of a first fluorescent probe configured to bind with a first target bioparticle and defining a first excitation wavelength range; and a second amount of a second fluorescent probe configured to bind with a second target bioparticle and defining a second excitation wavelength range, wavelengths within the second excitation wavelength range exceeding wavelengths within the first excitation wavelength range. The excitation source 154 is configured to: illuminate the detection region according to a first target excitation intensity corresponding to the first excitation wavelength range during a first detection period; and illuminate the detection region according to a second target excitation intensity corresponding to the second excitation wavelength range during a second detection period.

For example, the excitation source 154 can include: a first LED configured to illuminate the detection region at excitation wavelengths within the first excitation wavelength range; and a second LED configured to illuminate the detection region at excitation wavelengths within the second excitation wavelength range. A controller 190 of the reader 150 can then: activate the first LED and deactivate the second LED for a duration of the first detection period; and activate the second LED and deactivate the first LED for a duration of the second detection period. Alternatively, in another example, a user (e.g., a lab technician) may manually activate and deactivate the first and second LED prior to each detection period.

In the preceding implementation, the reader 150 can therefore be configured to: during the first detection period, detect a first optical signal (e.g., fluorescence) in a target emission wavelength range and representing presence of the first target bioparticle in fluid in the sample well 120; and, during the second detection period, detect a second optical signal (e.g., fluorescence) in the target emission wavelength range and representing presence of the second target bioparticle in fluid in the sample well 120.

### 7. Automated Sample Processing & Detection

In one implementation, the system 100 can be configured to autonomously: process samples within the cartridge 102; and detect presence of a set of target bioparticles within these samples based on detection of optical signals generated by fluorescent probes present in the set of samples.

In this implementation, the system 100 can include: a cartridge 102 including a set of sample wells arranged on the substrate 110; and a detection module 104 (e.g., an automated detection module). The detection module 104 can include: a cartridge receptacle 160 configured to receive the cartridge 102 and including a cartridge platform 162 (or “platform” 162)—defining a set of apertures 163 and configured to contact the lower surface 114 of the filter membrane 130 to support the cartridge 102 in a drain position—and a set of pump inlets 164 fluidly coupled to the array of apertures; a set of fluid dispensers 180 arranged above the cartridge receptacle 160 and configured to dispense metered volumes of fluid into the set of sample wells 120; and a set of pumps 170 coupled to the set of pump inlets 164 and configured to draw fluid and biological particulate from the inner surface 132 of the filter membrane 130 through the network of pores 136, and away from the outer surface 134 of the filter membrane 130; and a reader 150 arranged above the cartridge receptacle 160 and configured to detect a set of optical signals generated by fluid in the set of sample wells 120 responsive to excitation of the set of fluorescent probes. In this implementation, the reader 150 can include: an excitation source 154 configured to illuminate a detection region according to a target excitation wavelength; and a detector 152 (e.g., a photodetector) defining a field of view intersecting the detection region and configured to detect an optical signal—representing presence of the target bioparticle in a sample well—generated by fluid in the sample well, in the set of sample wells, located within the detection region, responsive to activation of the excitation source 154. The detection module 104 can also include a controller 190 configured to selectively trigger actuation of the set of fluid dispensers 180, the set of pumps 170, and the reader 150. Additionally, in one implementation, the controller can be configured to interpret presence of a set of target bioparticles in the set of sample wells 120 based on the set of optical signals detected by the reader 150.

The system 100 can therefore be configured to automatically: load sample wells on the substrate 110 with samples and/or reagent assigned to these detection samples—such as based on a cartridge 102 map defined for the cartridge 102 and defining a layout for the set of sample wells on the substrate 110—via actuation of the set of fluid dispensers 180; drain fluid and particulate from the set of sample wells 120 via actuation of the set of pumps 170; move the reader 150 (e.g., an XYZ plane and/or in a Z plane) and/or move the cartridge 102 (e.g., in an XY plane) to locate a particular sample well 120, in the set of sample wells 120, within the detection region defined by the reader 150, such as intersecting a field of view of a detector 152 (e.g., photodetector) of the reader 150; capture an optical signal generated by a fluid sample—loaded within the particular sample well 120—responsive to actuation of an excitation source 154 (e.g., a UV LED) arranged within and/or proximal the reader 150; and detect presence of one or more target bioparticles in the particular sample well 120 based on this optical signal.

### 7.1 Automated Process

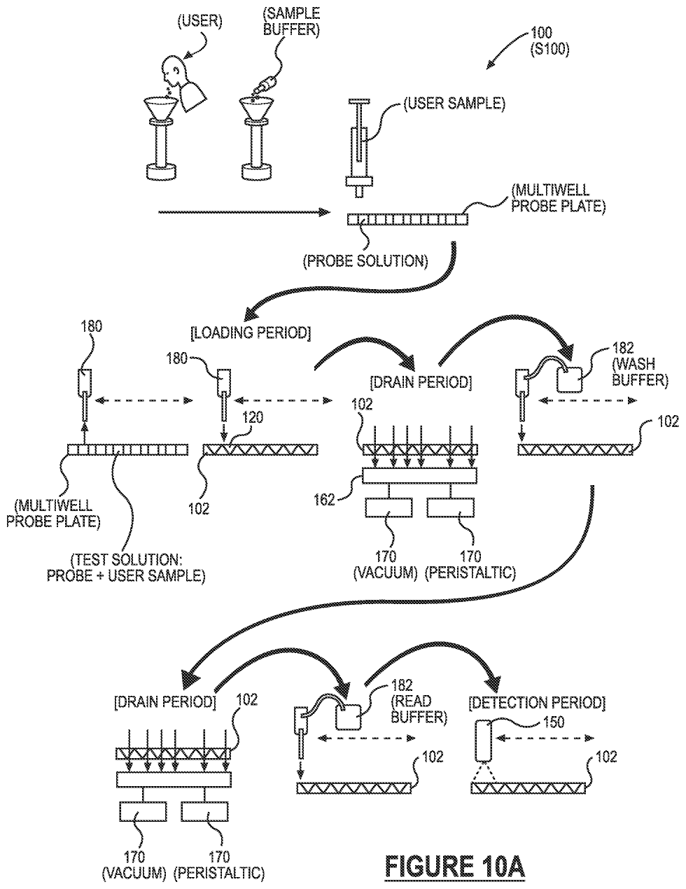

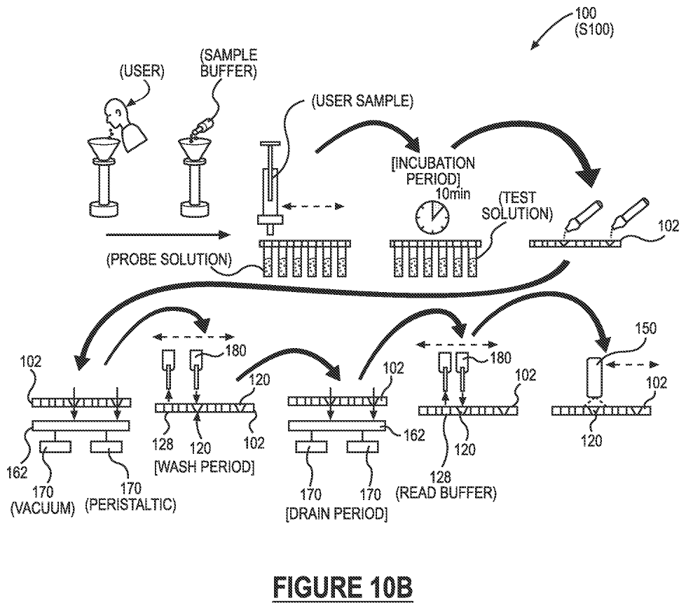

In particular, in this implementation, as shown in FIGS. 10A and 10B, the system 100 can be configured to: receive the cartridge within the cartridge receptacle including a platform configured to support the cartridge during draining of the set of sample wells and a lift plate configured to seat the cartridge on the platform and transiently raise the cartridge off the platform; locate the cartridge on the platform—fluidly coupled to a set of pumps configured to draw fluid out of the set of sample wells, through the corresponding filter membranes, and into a waste reservoir arranged below the platform; raise the cartridge off of the platform—via raising of the lift plate from a retracted position into an extended position—to receive volumes of a wash buffer and/or read buffer from the set of fluid dispensers, according to a particular assay and/or cartridge map defined for the cartridge, and/or in preparation for analysis of fluid within the set of sample wells via the reader 150 150.

For example, the system—such as via the controller 150 and/or a remote computer system interfacing with the system 100—can: at a first time, raise the lift plate to an extended position to receive the cartridge—each sample well in the cartridge preloaded with a test solution (e.g., a mixture of the probe solution and the user sample)—on the lift plate; during a first drain period, lower the lift plate to a retracted position to seat the cartridge on the platform and activate the set of pumps—such as a single vacuum pump or a vacuum pump paired with a peristaltic pump—to seal the lower surface of the cartridge to the platform and draw fluid through filter membranes of the set of sample wells into the waste reservoir; during a first dispense period succeeding the first drain period, raise the lift plate from the retracted position to the extended position and drive the set of fluid dispensers—according to a particular assay and/or cartridge map defined for the cartridge—to dispense metered volumes of a wash buffer into sample wells, in the set of sample wells; during a second drain period succeeding the wash period, lower the lift plate to the retracted position to seat the cartridge on the platform and activate the set of pumps to seal the lower surface of the cartridge to the platform to draw fluid through the set of filter membranes and into the waste reservoir; and, during a second dispense period succeeding the second drain period, raise the lift plate from the retracted position to the extended position and drive the set of fluid dispensers—according to the particular assay and/or cartridge map defined for the cartridge—to dispense metered volumes of a read buffer into sample wells, in the set of sample wells.

Finally, during a first detection period succeeding the second drain period, the system 100 can: raise the lift plate to the extended position; drive the reader 150 to a first reader 150 position, in a set of reader 150 positions, arranged above a first sample well, in the set of sample wells, such as according to the cartridge map; activate an excitation source configured to illuminate the first sample well; and activate the detector 152 (e.g., photodetector)—such as including an optical sensor configured to capture a single image of a detection region encompassing the first sample well—to record a first optical signal, in a set of optical signals, generated by fluid in the first sample well responsive to activation of the excitation source. The computer system and/or controller can then interpret presence of a first target bioparticle, in set of target bioparticles, present in the first sample well based on the first optical signal. During the detection period, the system 100 can repeat this process to record optical signals from each sample well on the cartridge, and thereby enable detection of the set of target bioparticles across the set of sample wells within the cartridge.

Additionally and/or alternatively, in another example, as shown in FIG. 10A, the system 100 can be configured to load the test solution into the set of sample wells. For example, during an initial loading period, the system 100 can: raise the lift plate to the extended position to receive the cartridge on the lift plate; and drive the set of fluid dispensers (e.g., according to the cartridge map defined for the cartridge) to draw a metered volume of each test sample, in a set of test samples, loaded in sample wells of a secondary cartridge—each sample well, in the secondary cartridge, loaded with a user sample mixed with the probe solution—and dispense the metered volume of the test sample into a particular sample well, in the set of sample wells in the cartridge, according to the cartridge map. Then, during an initial drain period succeeding the initial loading period and preceding the first time, the system 100 can: lower the lift plate to the retracted position to seat the cartridge on the platform; and activate the set of pumps to seal the lower surface of the cartridge to the platform and draw fluid through filter membranes of the set of sample wells into the waste reservoir, thereby draining fluid and biological particulate out of the sample wells and isolating the target complex (e.g., an amount of the target complex) on the filter membrane within the sample well. de

### 7.2 Controller

The detection module 104 can include a controller 190 including a set of electronics and configured to: selectively actuate components of the detection device to enable autonomous execution of Blocks of the method S100 to process a set of samples—loaded in the set of sample wells 120 on the substrate 110—and detect optical signals emitted by fluid retained in the set of sample wells 120; and interpret presence of a set of target bioparticles in fluid in the set of sample wells 120—and thereby in the initial user sample collected from the user (e.g., a human patient)—based on these optical signals.

In one implementation, the system 100 can include a communication module (e.g., a wireless communication module) coupled to the controller 190 and configured to communicate updates regarding functioning of the system 100 to a remote computer system 100. For example, the controller 190 can be configured to access a set of signals recorded by a set of sensors 184 installed in the detection module 104 and configured to monitor loading, draining, and/or positioning of the set of sample wells 120. The controller 190 can interpret errors—such as incomplete draining of a particular sample well 120 or sample wells 120, loading of a user sample into an incorrect sample well 120, leakage of fluid from a sample well 120 into a neighboring well, etc.—based on the set of signals recorded by the set of sensors 184. The communication module—coupled to the controller 190—can then communicate these errors to the remote computer system 100.

### 7.3 Drain Subsystem

The detection module 104 can define a drain subsystem configured to drain contents of sample wells in the cartridge 102 into the waste reservoir 172. In particular, the detection module 104 can include a set of pumps—fluidly coupled to the set of apertures 163 on the platform 162—configured to draw fluid in a sample well 120 through the filter membrane 130 and off of the outer surface 134 of the filter membrane 130. Further, the cartridge receptacle 160 can include: the platform 162 configured to receive and support the cartridge and defining a set of apertures 163; and a set of pump inlets 164 fluidly coupled to the set of apertures 163 arranged on the platform 162 and coupled to the set of pumps 170, such that fluid—including air and/or liquid flowing off of the filter membrane 130—can be drawn through the set of apertures 163 in the platform 162 responsive to activation of the set of pumps 170.

The cartridge receptacle 160 can therefore cooperate with the set of pumps 170—such as including a single vacuum pump and/or a vacuum pump in combination with a secondary or “liquid-draining” pump (e.g., a peristaltic pump)—to form the drain subsystem. Further, the detection module 104 can include a waste reservoir 172—such as arranged below the platform 162—configured to collect fluid flowing off of the outer surface 134 of the filter membrane 130.

In particular, the cartridge receptacle 160 can include: the platform 162 (“or drain platform”) defining a set of apertures 163—such as a first subset of apertures 163 configured for air flow to draw the cartridge against the platform and a second subset of apertures 163 configured to disperse fluid (e.g., liquid fluid) from the filter membrane toward the waste reservoir 172—and configured to support the cartridge in the drain position; and a set of pump inlets 164 fluidly coupled to apertures in the set of apertures 163 (e.g., the first and/or second subset of apertures 163). The detection module 104 can further include the set of pumps 170 coupled to the set of pump inlets 164 and configured to draw fluid and biological particulate from the inner surface 132 of the filter membrane 130 through the network of pores 136, and away from the outer surface 134 of the filter membrane 130.

In one implementation, the set of pumps 170 can include a vacuum pump 170—configured to couple to a pump inlet 164—configured to draw air through the set of apertures 163 on the platform 162 to generate a vacuum between surfaces of the cartridge 102—such as the outer surface 134 of the filter membrane 130—and surfaces of the platform 162, and thereby draw fluid through the network of pores 136 and off the outer surface 134 of the filter membrane 136. In this implementation, the detection module 104 can include the waste reservoir 172 arranged below the platform 162 and configured to collect fluid flowing off the outer surface 134 of the filter membrane 130.

Additionally, in this implementation, the platform 162 can define: a base surface; a set of pedestals—extending upward from the base surface—configured to contact the outer surface of each filter membrane on the cartridge (e.g., in the drain position), such that the cartridge seats above the base surface (e.g., by 1 millimeter, 2 millimeters, 5 millimeters) and on the set of pedestals. Further, the set of apertures 163 can extend through the set of pedestals, such that the outer surface 134 of each filter membrane 130—seated on a pedestal in the set of pedestals—is drawn against the pedestal during actuation of the vacuum pump 170, thereby forming a vacuum between the outer surface 134 of the filter membrane 130 and a surface of the pedestal. The set of apertures 163 can then direct fluid flowing off the outer surface 134 into the waste reservoir 172.

In particular, each pedestal, in the set of pedestals, can include a subset of apertures 163, in the set of apertures 163, extending between a surface of the pedestal—contacting a corresponding filter membrane on the cartridge—and a pump inlet 164 in the set of pump inlets 164. Then, during draining, the system 100 can activate the vacuum pump—coupled to the pump inlet 164—to draw air through the subset of apertures 163 integrated into each pedestal, in the set of pedestals, to suction the filter membrane against the pedestal, and therefore draw fluid from the sample well, through the filter membrane, and off of the outer surface of the filter membrane. Each pedestal, in the set of pedestals, can further include a second subset of apertures 163, in the set of apertures 163, configured to collect fluid flowing off of the outer surface of the filter membrane, and into the waste reservoir 172. Additionally and/or alternatively, each aperture, in the set of apertures 163, can be configured to enable both air and liquid flow through the aperture.

For example, the platform 162 can define: a base surface; a set of pedestals extending from the base surface and defining a set of apertures 163, each pedestal, in the set of pedestals, defining a concave surface configured to contact a corresponding filter membrane 130 arranged across a lower opening 114 of a sample well 120, in a set of sample wells 120, in the cartridge 102; and a pump inlet 164 fluidly coupled to the set of apertures 163. In particular, the set of pedestals can include a first pedestal—defining a first set of apertures 163 fluidly coupled to the pump inlet and defining a concave surface configured to contact a first filter membrane 130, in the set of filter membranes 130, arranged below a first sample well 120, in the set of sample wells 120, of the cartridge 102. In this example, the cartridge 102 can be configured to seat on the platform 162, such that each filter membrane 130, in the set of filter membrane 130, is aligned with a pedestal in the set of pedestals on the platform 162 (e.g., with the lower surface 114 of the substrate 110 seated on a lip of the pedestal). Then, during draining, the system 100 can activate the vacuum pump—coupled to the pump inlet 164—to: draw air through the first set of apertures 163 to draw surfaces of the cartridge 120 (e.g., the lower surface 114 and the outer surface 134) against the first pedestal and/or toward the concave surface of the first pedestal to generate a vacuum; and draw fluid off of the outer surface 134 of the first filter membrane 130, through the first set of apertures 163, and toward the waste reservoir 172 arranged beneath the platform 162. In particular, the concave surface of the first pedestal can be configured to direct fluid toward one or more apertures arranged in the pedestal. The set of apertures 163 can then direct fluid (e.g., liquid fluid) toward the waste reservoir arranged beneath the platform 162. The set of pedestals can similarly include additional pedestals configured to receive a corresponding filter membrane, in the set of filter membranes 130 on the cartridge, for draining of a corresponding sample well in the set of sample wells 120.

Further, in the preceding example, the system 100 can include a filter arranged within an air outlet—fluidly coupling the pump inlet 164 and/or vacuum pump 170 to the set of apertures 163—configured to collect fluid (e.g., liquid fluid) and biological particulate flowing toward the vacuum pump 170 and prevent drawing of liquid fluid and biological particulate into the vacuum pump 170. Once draining of the set of sample wells is complete, the system 100 can drive the cartridge 102 to the extended position (e.g., via the lift plate) in preparation for loading with additional fluid—according to a particular assay—and/or in preparation for detection via the reader 150.

Additionally and/or alternatively, in another implementation, the set of pumps 170 can include: a vacuum pump fluidly coupled (e.g., via an airway) to a first subset of apertures 163, in the set of apertures 163, and configured to apply a vacuum between the cartridge receptacle 160 and the cartridge 102 to draw fluid and biological particulate from the inner surface 132 of the filter membrane 130 through the network of pores 136; and a fluid-draining pump—such as a peristaltic pump—fluidly coupled to a second subset of apertures 163, in the set of apertures 163, via a set of drain tubes extending through the second subset of apertures 163 and contacting the lower surface 114 of the filter membrane 130 (e.g., for each sample well 120). The vacuum pump can therefore be configured to generate a seal between the cartridge receptacle 160 and the cartridge 102, thereby promoting flow of fluid through the network of pores 136 and onto the outer surface 134 of the filter membrane 130. The secondary pump can draw fluid present on the outer surface 134 of the filter membrane 130—due to the seal generated by the vacuum pump—into the set of drain tubes and through the secondary pump for removal from the cartridge 102. In particular, in this implementation, the secondary pump can be coupled to a waste reservoir 172 for collection of fluid drained from the cartridge 102

### 7.3.1 Lift Mechanism

The detection module 104 can be configured to locate the cartridge 102 in a retracted position (or “drain position”)—in which the cartridge 102 is seated on the platform 162—and an extended position (or “raised position”) in which the cartridge 102 is lifted off of the platform 162.

In one implementation, as shown in FIG. 8, the cartridge receptacle 160 can include a set of springs 166—such as coupled to the base surface of the platform 162—configured to contact the lower surface 112 of the substrate to support the cartridge 102 and transiently locate the cartridge 102 in the retracted and/or extended positions. The set of springs 166 can thus: transition between the extended position to the retracted position to seat the cartridge 102 on the platform 162—and/or on the set of pedestals of the platform 162—in preparation for a drain cycle; and transition between the retracted position to the extended position to lift the cartridge 102 off of the platform 162 in preparation for a loading cycle and/or detection cycle. Further, in this implementation, the detection module 104 can include a compression mechanism configured to enable compression and decompression of the set of springs 166 accordingly. For example, the detection module 104 can include a set of dispenser tips—coupled to a dispenser actuator—configured to: and exert a downward force on the cartridge 102 to compress the set of springs 166 and thereby locate the set of springs 166 and cartridge 102 in the retracted position; release the cartridge—via removal the downward force—to decompress the set of springs 166 and thereby locate the set of springs 166 and cartridge 102 in the extended position.

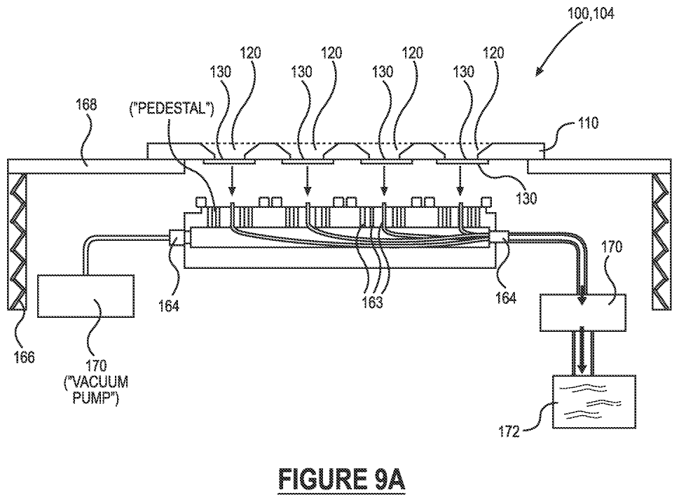

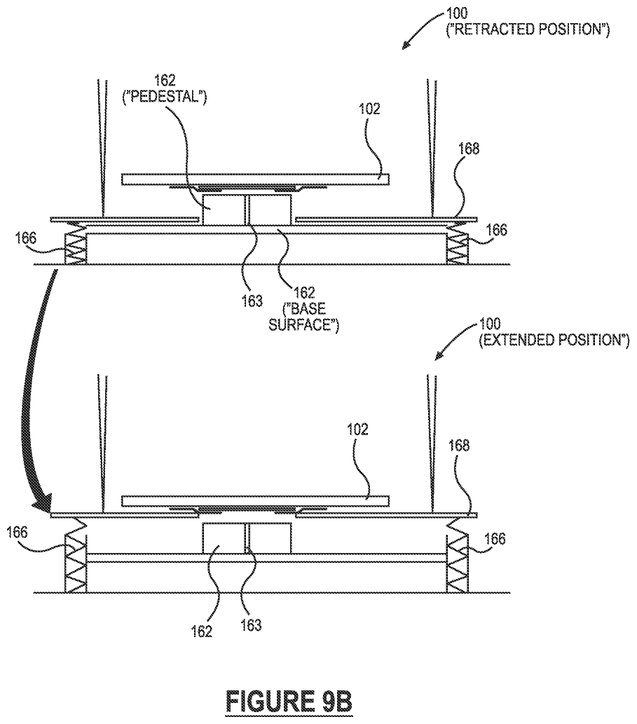

Alternatively, in another implementation, as shown in FIGS. 9A and 9B, the cartridge receptacle 160 can include: a set of springs 166 configured to transition between the extended and retracted positions; a lift plate 168 seated on the set of springs 166 and configured to transiently contact the lower surface 114 of the substrate 110 and/or cartridge 102—responsive to transition of the set of springs 166 toward the extended position—to lift the cartridge 102 off of the platform 162 and locate the cartridge 102 in the extended position; and a compression mechanism configured to enable compression and decompression of the set of springs 166 accordingly. In this implementation, the set of springs 166 can thus transition between the retracted position to the extended position to: raise the lift plate toward the lower surface 114 and seat the cartridge 102 on the lift plate; and locate the cartridge 102—seated on the lift plate—in the extended position in preparation for a loading cycle and/or detection cycle. Further, the set of springs can transition between the extended position to the retracted position to lower the lift plate and seat the cartridge on the platform 162 in the retracted position in preparation for a draining cycle.

### 7.4 Fluid Dispensers

The detection module 104 can include a set of fluid dispensers 180 arranged above the cartridge receptacle 160 and configured to dispense metered volumes of fluid into the set of sample wells 120. In particular, the controller 190 can selectively actuate the set of fluid dispensers 180 to dispense volumes of a test solution, user sample, probe solution, wash buffer, and/or read buffer into the set of sample wells according to a predefined assay and/or cartridge map.

In one implementation, as shown in FIG. 10A, the set of fluid dispensers 180 can be fluidly coupled to a set of reagent reservoirs 182 loaded with reagents for dispensation into the set of sample wells. For example, the detection module 104 can include: a first reagent reservoir 182 loaded with a volume of a wash buffer; a second reagent reservoir 182 loaded with a volume of a read buffer; a first fluid dispenser 180—fluidly coupled to the first reagent reservoir 182—configured to dispense metered volumes of the wash buffer into the set of sample wells; and a second fluid dispenser 180—fluidly coupled to the second reagent reservoir 182—configured to dispense metered volumes of the read buffer into the set of sample wells 120. Alternatively, in another implementation, as shown in FIG. 10B, the set of fluid dispensers 180 can be configured to: draw volumes of fluid from a set of reagent wells 128 integrated within the cartridge 102 and loaded with volumes of reagents (e.g., read buffer and/or wash buffer); and dispense metered volumes of fluid—collected from the set of reagent wells 128—into the set of sample wells 120.

In one variation, the detection module 104 includes a set of sensors 184 coupled to the set of fluid dispensers 180. In particular, the set of sensors 184 can include: a first subset of sensors 184 configured to record location or position of fluid dispensation on a cartridge 102, such as a position of a particular sample well 120 (e.g., within the cartridge 102) located beneath a sensor in the first subset of sensors 184; and a second subset of sensors configured to record a fluid fill level of each sample well 120 on the cartridge 102, such as before and/or after dispensation of fluid into the sample well 120. Additionally and/or alternatively, the diagnostic model 104 can include a set of sensors 184 decoupled from the set of fluid dispensers 180. For example, the detection module 104 can include a sensor 184, in the set of sensors 184, configured to record a fill level of each sample well 120 on the cartridge 102 during a drain cycle. In this example, the controller 190 can be configured to: access the fill level recorded by the sensor 184; and selectively actuate the set of pumps 170 based on the fill level recorded by the sensor.

### 7.5 Device Actuators

The controller 190 can be configured to coordinate motion of the set of fluid dispensers 180, the reader 150 and/or the cartridge 102—such as via motion of the cartridge receptacle 160—to locate a particular sample well 120, in the set of sample wells 120, in a particular position.

In one implementation, the controller 190 can access a cartridge 102 map—defining a layout of sample wells 120 within a cartridge 102—defined for the cartridge 102. For example, a user (e.g., a lab technician) may assemble the cartridge 102 map during an initial setup period and upload the cartridge 102 map to a remote computer system 100. Alternatively, in this example, the remote computer system 100 can automatically assemble the cartridge 102 map based on a known quantity of user samples, a type of user samples, and/or a particular target bioparticle or group of target bioparticles assigned to the cartridge 102. In each of these examples, the communication module can receive the cartridge 102 map from the remote computer system 100 and upload the cartridge 102 map onto the controller 190.

The detection module 104 can include a set of actuators configured to move components of the detection module 104 according to instructions output by the controller 190. For example, the detection module 104 can include: a cartridge 102 actuator configured to move the cartridge 102 in an XY plane to locate a particular sample well 120, in the set of sample wells 120, in a particular position (e.g., relative the reader 150, relative a fluid dispenser in the set of fluid dispensers 180); a reader 150 actuator configured to move the reader 150 in an Z plane and/or in an XYZ plane to locate the reader 150 proximal a sample well 120, in the set of sample wells 120, such that the sample well 120 is located within the detection region defined by the reader 150; and/or a dispenser actuator configured to move the set of fluid dispensers 180 in an XY plane and/or in an XYZ plane to locate a fluid dispenser and/or the set of fluid dispensers 180 above a particular sample well 120, in the set of sample wells 120, and/or above a particular subset of sample wells 120 in the set of sample wells 120. The controller 190 can thus coordinate motion of the set of fluid dispensers 180, the reader 150 and/or the cartridge 102 by selectively triggering actuation of the set of actuators according to the cartridge 102 map defined for the cartridge 102.

The system loos and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other system loos and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

I claim:

1. A system comprising:
a cartridge comprising:
a substrate defining an upper surface and a lower surface; and
a set of sample wells integrated into the substrate, each sample well in the set of sample wells:
defining an upper opening, in a set of upper openings, arranged on the upper surface;
defining a lower opening, in a set of lower openings, arranged on the lower surface; and
comprising a filter membrane, in a set of filter membranes, coupled to the lower surface, extending across the lower opening, and defining:
an inner surface facing the sample well;
an outer surface; and
a network of pores extending between the inner surface and the outer surface and configured to promote transfer of fluid and biological particulate from the inner surface to the outer surface and inhibit passage of a target complex through the filter membrane, the target complex comprising a fluorescent probe, in a set of fluorescent probes, bound to a target bioparticle, in a set of target bioparticles; and
a detection module comprising:
a cartridge receptacle configured to receive the cartridge and comprising:
a platform defining a set of apertures and configured to support the cartridge in a drain position; and
a set of pump inlets fluidly coupled to the set of apertures;
a set of fluid dispensers arranged above the cartridge receptacle and configured to dispense metered volumes of fluid into the set of sample wells;
a set of pumps coupled to the set of pump inlets and configured to draw fluid and biological particulate from the inner surface of the filter membrane through the network of pores, and away from the outer surface of the filter membrane;
a reader arranged above the cartridge receptacle and configured to detect a set of optical signals generated by fluid in the set of sample wells responsive to excitation of the set of fluorescent probes, the set of optical signals representing presence of the set of target bioparticles in the set of sample wells; and
a controller configured to selectively trigger actuation of the set of fluid dispensers, the set of pumps, and the reader.

2. The system of claim 1:
wherein the set of sample wells comprises:
a first sample well:
defining a first upper opening, in the set of upper openings, arranged on the upper surface;
defining a first lower opening, in the set of lower openings, arranged on the lower surface; and
comprising a first filter membrane, in the set of filter membranes, coupled to the lower surface and extending across the first lower opening;
a second sample well:
defining a second upper opening, in the set of upper openings, arranged on the upper surface;
defining a second lower opening, in the set of lower openings, arranged on the lower surface; and
comprising a second filter membrane, in the set of filter membranes, coupled to the lower surface and extending across the second lower opening; and
wherein the reader is configured to:
detect a first optical signal, in the set of optical signals, generated by fluid in the first sample well during a first detection period; and
detect a second optical signal, in the set of optical signals, generated by fluid in the second sample well during a second detection period; and
wherein the controller is configured to:
coordinate motion of the reader to locate the first sample well within a detection region, intersecting a field of view of an optical sensor of the reader, during the first detection period; and
coordinate motion of the reader to locate the second sample well within the detection region during the second detection period.

3. The system of claim 2:
wherein the first sample well is configured to receive a first user sample, derived from a first user, and a first subvolume of a probe solution comprising a first fluorescent probe, in the set of fluorescent probes, configured to bind with a first target bioparticle, in the set of target bioparticles, to form a first target complex;
wherein the second sample well is configured to receive a second user sample, derived from a second user, and a second subvolume of the probe solution; and
wherein the controller is configured to:
interpret presence of the first bioparticle in the first sample well based on the first optical signal generated responsive to excitation of the first fluorescent probe in the first sample well; and
interpret presence of the first bioparticle in the second sample well based on the second optical signal generated responsive to excitation of the first fluorescent probe in the second sample well.

4. The system of claim 2:
wherein the first sample well is configured to receive a first subvolume of a user sample, derived from a first user, and a first probe solution comprising a first fluorescent probe, in the set of fluorescent probes, configured to bind with a first target bioparticle, in the set of target bioparticles, to form a first target complex;
wherein the second sample well is configured to receive a second subvolume of the user sample and a second probe solution comprising a second fluorescent probe, in the set of fluorescent probes, configured to bind with a second target bioparticle, in the set of target bioparticles, to form a second target complex; and
wherein the controller is configured to:
interpret presence of the first bioparticle in the first sample well based on the first optical signal generated responsive to excitation of the first fluorescent probe in the first sample well; and
interpret presence of the second bioparticle in the second sample well based on the second optical signal generated responsive to excitation of the second fluorescent probe in the second sample well.

5. The system of claim 1:
wherein the platform comprises:
a base surface; and
a set of pedestals extending from the base surface and configured to contact the outer surface of the set of filter membranes in the drain position;
wherein the set of apertures extend through the set of pedestals;
wherein the set of pumps comprises a vacuum pump coupled to a first pump inlet, in the set of pump inlets, and configured to draw air through the set of apertures to apply a vacuum between the set of pedestals and the set of filter membranes to draw fluid and biological particulate from the inner surface, through the network of pores, and off of the outer surface of each filter membrane in the set of filter membranes; and
further comprising a waste reservoir arranged below the base surface and configured to collect fluid and biological particulate released from the outer surface of the filter membrane.

6. The system of claim 1:
further comprising a waste reservoir configured to collect fluid flowing off of the outer surface of the filter membrane; and
wherein the set of pumps comprises:
a vacuum pump:
fluidly coupled to a first subset of apertures in the set of apertures; and
configured to apply a vacuum between the cartridge receptacle and the cartridge to draw fluid and biological particulate from the inner surface of the filter membrane, through the network of pores, onto the outer surface of the filter membrane;
a secondary pump:
fluidly coupled to a second subset of apertures, in the set of apertures, via a set of drain tubes extending through the second subset of apertures and contacting the outer surface of the filter membrane; and
configured to draw fluid off of the filter membrane, through the secondary pump, and into the waste reservoir.

7. The system of claim 1:
wherein the detection module further comprises a set of sensors configured to record a set of fluid fill levels of the set of sample wells; and
wherein the controller is configured to selectively actuate the set of pumps based on the set of fluid fill levels.

8. The system of claim 7:
wherein the set of sensors is configured to record a set of fill positions on the cartridge during dispensation of fluid from fluid dispensers, in the set of fluid dispensers, into the set of sample wells;
wherein the controller is configured to detect errors in fill position, in the set of fill positions, based on a cartridge map defined for the cartridge and defining a layout of the set of sample wells on the cartridge; and
wherein the detection module further comprises a communication module coupled to the controller and configured to communicate errors to a remote computer system.

9. The system of claim 1:
wherein the detection module further comprises:
a first reagent reservoir fluidly coupled to the set of fluid dispensers and loaded with a volume of a wash buffer; and
a second reagent reservoir fluidly coupled to the set of fluid dispensers and loaded with a volume of a read buffer; and
wherein the controller is configured to selectively actuate the set of fluid dispensers to:
dispense subvolumes of the wash buffer from the first reagent reservoir into sample wells in the set of sample wells; and
and dispense subvolumes of the read buffer from the second reagent reservoir into sample wells in the set of sample wells.

10. The system of claim 1:
wherein the cartridge further comprises a set of reagent wells integrated into the substrate and comprising:
a first subset of reagent wells configured to store a volume of a wash buffer; and
a second subset of reagent wells configured to store a volume of a read buffer; and
wherein the set of fluid dispensers are configured to:
withdraw subvolumes of the wash buffer from reagent wells, in the first subset of reagent wells, during a wash period;
dispense metered volumes of the wash buffer, withdrawn from the first subset of reagent wells, into the set of sample wells during the wash period;
withdraw subvolumes of the read buffer from reagent wells, in the second subset of reagent wells, during a detection period succeeding the wash period; and
dispense metered volumes of the read buffer, withdrawn from the second subset of reagent wells, into the set of sample wells during the detection period;
wherein the controller is configured to coordinate motion of the set of fluid dispensers during the wash period and the detection period according to a cartridge map defined for the cartridge.

11. The system of claim 1:
wherein the reader comprises:
an optical sensor:
defining a field of view intersecting a detection region;
configured to record a first optical signal, in the set of optical signals, generated by fluid in a first sample well, in the set of sample wells, located within the detection region during a first detection period;
configured to record a second optical signal, in the set of optical signals, generated by fluid in a second sample well, in the set of sample wells, located within the detection region during a second detection period;
an excitation source configured to illuminate the detection region according to a target excitation wavelength, defined by a particular fluorescent probe, in the set of fluorescent probes, loaded in a detection well, in the set of detection wells, located in the detection region; and
wherein the controller is configured to:
selectively trigger activation of the excitation source and the optical sensor;
coordinate motion of the reader to locate a particular sample well, in the set of sample wells, within the detection region.

12. The system of claim 11:
wherein the reader further comprises a set of optical filters, each optical filter, in the set of optical filters, corresponding to a target emission wavelength in a set of target emission wavelengths, and configured to transiently install in a filter slot arranged between the optical sensor and the detection region; and
wherein the controller is configured to coordinate activation of a particular emission filter, in the set of filters, in the filter slot based on the particular fluorescent probe loaded in the detection well located in the detection region.

13. The system of claim 1:
wherein the reader comprises:
an optical sensor:
defining a field of view intersecting a detection region;
configured to record a first optical signal, in the set of optical signals, generated by a first fluorescent probe, in the set of fluorescent probes, present in a first sample well, in the set of sample wells, located within the detection region, during a first detection period, the first fluorescent probe defining a first target excitation wavelength;
configured to record a second optical signal, in the set of optical signals, generated by a second fluorescent probe, in the set of fluorescent probes, during a second detection period, the second fluorescent probe defining a second target excitation wavelength;
an excitation source configured to illuminate the detection region and comprising:
a first LED defining a first excitation wavelength corresponding to the first target excitation wavelength;
a second LED defining a second excitation wavelength corresponding to the second target excitation wavelength;
wherein the controller is configured to:
trigger activation of the first LED during the first detection period;
trigger activation of the second LED during the second detection period;
trigger activation of the optical sensor during the first detection period and the second detection period.

14. The system of claim 1:
wherein the set of sample wells comprises:
a first sample well loaded with a first test solution comprising a first volume of a user sample mixed with a first probe solution comprising an amount of a first fluorescent probe, in a set of fluorescent probes, configured to bind to a first target bioparticle in a set of target bioparticles; and
a second sample well loaded with a second test solution comprising a second volume of the user sample mixed with a second probe solution comprising an amount of a second fluorescent probe, in the set of fluorescent probes, configured to bind to a second target bioparticle in the set of target bioparticles;
wherein the reader is configured to detect:
a first optical signal, in the set of optical signals, generated by fluid in the first sample well responsive to excitation of the first fluorescent probe; and
a second optical signal, in the set of optical signals, generated by fluid in the second sample well responsive to excitation of the second fluorescent probe.

15. The system of claim 14:
wherein the first sample well is loaded with the first test solution comprising the first volume of the user sample mixed with the first probe solution comprising the amount of the first fluorescent probe comprising:
a first antibody configured to bind to the first target bioparticle; and
a first fluorescent tag bound to the first antibody and configured to generate the first optical signal responsive to excitation via the excitation source;
wherein the second sample well is loaded with the second test solution comprising the second volume of the user sample mixed with the second probe solution comprising the amount of the second fluorescent probe comprising:
a second antibody configured to bind to the second target bioparticle; and
a second fluorescent tag bound to the second antibody and configured to generate the second optical signal responsive to excitation via the excitation source.

16. A system comprising:
a cartridge comprising:
a substrate defining an upper surface and a lower surface; and
a first sample well:
defining an upper opening arranged on the upper surface;
defining a lower opening arranged on the lower surface; and
comprising a filter membrane coupled to the lower surface, extending across the lower opening, and defining:
an inner surface facing the first sample well;
an outer surface; and
a network of pores extending between the inner surface and the outer surface and configured to promote transfer of fluid and biological particulate from the inner surface to the outer surface and inhibit passage of a target complex through the filter membrane, the target complex comprising a fluorescent probe bound to a target bioparticle; and
a detection module comprising:
a cartridge receptacle configured to receive the cartridge and comprising:
a platform defining a set of apertures and configured to contact the lower surface to support the cartridge in a drain position; and
a set of pump inlets fluidly coupled to the array of apertures;
a set of fluid dispensers arranged above the cartridge receptacle and configured to dispense metered volumes of fluid into the first sample well;
a set of pumps coupled to the set of pump inlets and configured to draw fluid and biological particulate from the inner surface of the filter membrane through the network of pores, and away from the outer surface of the filter membrane;
a reader arranged above the cartridge receptacle and configured to detect an optical signal generated by fluid in the first sample well responsive to excitation of the fluorescent probe; and
a controller configured to:
selectively trigger actuation of the set of fluid dispensers, the set of pumps, and the reader according to a predefined assay; and
interpret presence of the target bioparticle in the first sample well based on the optical signal.

17. The system of claim 16:
wherein the cartridge comprises a second sample well:
defining a second upper opening arranged on the upper surface;
defining a second lower opening arranged on the lower surface; and
comprising a second filter membrane coupled to the lower surface, extending across the second lower opening, and defining:
a second inner surface facing the second sample well;
a second outer surface; and
a second network of pores extending between the second inner surface and the second outer surface and configured to promote transfer of fluid and biological particulate from the second inner surface to the second outer surface and inhibit passage of a second target complex through the filter membrane, the second target complex comprising a second fluorescent probe bound to a second target bioparticle;
wherein the set of fluid dispensers is configured to dispense metered volumes of fluid into the first sample well and the second sample well;
wherein the pump is configured to apply a vacuum between the cartridge receptacle and the cartridge to draw fluid and biological particulate from the inner surface of the filter membrane through the network of pores;
wherein the reader is configured to:
detect afirstopticalsignalgeneratedbyfluidinthefirstsamplewellresponsive to excitation of the fluorescent probe via an excitation source during a first detection period; and
detect a second optical signal generated by fluid in the second sample well responsive to excitation of the second fluorescent probe via the excitation source during a second detection period; and
wherein the controller is configured to coordinate motion of the reader to:
locate the sample well within a detection region defined by the reader during the first detection period;
interpret presence of the target bioparticle in the first sample well based on the optical signal;
locate the second sample well within the detection region during the first detection period; and
interpret presence of the second target bioparticle in the second sample well based on the second optical signal.

18. The system of claim 16:
wherein the cartridge comprises a second sample well:
defining a second upper opening arranged on the upper surface;
defining a second lower opening arranged on the lower surface; and
comprising a second filter membrane coupled to the lower surface, extending across the second lower opening, and defining:
a second inner surface facing the second sample well;
a second outer surface; and
a second network of pores extending between the second inner surface and the second outer surface and configured to promote transfer of fluid and biological particulate from the second inner surface to the second outer surface and inhibit passage of the target complex through the filter membrane;
wherein the set of fluid dispensers is configured to dispense metered volumes of fluid into the first sample well and the second sample well;
wherein the pump is configured to apply a vacuum between the cartridge receptacle and the cartridge to draw fluid and biological particulate from the inner surface of the filter membrane through the network of pores;
wherein the reader is configured to:
detect a first optical signal generated by fluid in the sample well responsive to excitation of the fluorescent probe via an excitation source during a first detection period; and
detect a second optical signal generated by fluid in the second sample well responsive to excitation of the fluorescent probe via the excitation source during a second detection period; and
wherein the controller is configured to coordinate motion of the reader to:
locate the sample well within a detection region defined by the reader during the first detection period;
interpret presence of the target bioparticle in the first sample well based on the optical signal;
locate the second sample well within the detection region during the first detection period; and
interpret presence of the target bioparticle in the second sample well based on the second optical signal.

19. The system of claim 16:
wherein the sample well is configured to receive a test solution comprising a user sample mixed with a probe solution comprising an amount of the fluorescent probe configured to bind with the target bioparticle to form the target complex, the fluorescent probe of a first size less than a second size of the target complex; and
wherein pores, in the network of pores, exhibit sizes within a target pore size range, sizes within the target pore size range exceeding the first size and falling below the second size.

20. A system comprising:
a cartridge comprising:
a substrate defining an upper surface and a lower surface; and
a set of sample wells, each sample well, in the set of sample wells:
defining an upper opening arranged on the upper surface;
defining a lower opening arranged on the lower surface; and
comprising a filter membrane coupled to the lower surface, extending across the lower opening, and defining:
an inner surface facing the sample well;
an outer surface; and
a network of pores extending between the inner surface and the outer surface and configured to promote transfer of fluid and biological particulate from the inner surface to the outer surface and inhibit passage of a target complex through the filter membrane, the target complex comprising a fluorescent probe bound to a target bioparticle; and
a detection module comprising:
a cartridge receptacle configured to receive the cartridge and defining a set of apertures;
a set of fluid dispensers arranged above the cartridge receptacle and configured to dispense metered volumes of fluid into the first sample well;
a set of pumps fluidly coupled to the set of apertures and configured to draw fluid and biological particulate from the inner surface of the filter membrane through the network of pores, and away from the outer surface of the filter membrane of each sample well in the set of sample wells; and
a reader arranged above the cartridge receptacle and comprising:
an excitation source configured to illuminate a detection region according to a target excitation wavelength; and
a detector defining a field of view intersecting the detection region and configured to detect an optical signal generated by fluid in a first sample well, in the set of sample wells, located within the detection region, responsive to activation of the excitation source, the optical signal representing presence of the target bioparticle in the first sample well.
